(19) United States
(12) Patent Application Publication
(10) Pub. No.: US 2019/0060287 A1
(43) Pub. Date: Feb. 28, 2019

(54) TIZANIDINE FORMULATIONS

(71) Applicant: ADARE PHARMACEUTICALS, INC., Lawrenceville, NJ (US)

(72) Inventors: Gopi M. VENKATESH, Vandalia, OH (US); Craig G. KRAMER, New Lebanon, OH (US); Chinmay MAHESHWARI, Mason, OH (US); Peng GUO, Beavercreek, OH (US); Jin-Wang LAI, Springboro, OH (US)

(21) Appl. No.: 16/114,887

(22) Filed: Aug. 28, 2018

Related U.S. Application Data
(60) Provisional application No. 62/551,084, filed on Aug. 28, 2017.

(51) Int. Cl. A61K 31/433; A61K 9/48; A61K 9/16; A61P 25/14; A61P 21/02

(52) U.S. Cl. CPC A61K 31/433; A61K 9/4808; A61K 9/167; A61P 21/02; A61K 9/1652; A61K 9/1635; A61P 25/14; A61K 9/1623

(57) ABSTRACT

The disclosure is directed to pharmaceutical compositions comprising of Tizanidine or a pharmaceutically acceptable salt thereof which controllably modulate the onset of at least one secondary effect associated with Tizanidine administration. In embodiments, the pharmaceutical compositions comprise at least three components with different release characteristics: immediate release component; a first time, pulsatile release component; and a second population of time, pulsatile release component. The release characteristics of the pharmaceutical composition are such that the bedtime blood plasma concentrations control spasticity and allow the patient to have quality sleep, and the daytime blood plasma concentrations controls spasticity without unwanted secondary such as somnolence or sedation.

### CROSS REFERENCE TO RELATED APPLICATIONS

This application claims priority to U.S. Provisional Application No. 62/551,084, filed on Aug. 28, 2017, which is incorporated by reference herein in its entirety.

### FIELD OF THE DISCLOSURE

The present disclosure relates to a pharmaceutical composition comprising at least one drug for once-daily oral administration, which provides a primary therapeutic effect associated with the at least one drug over a 24 hour period and controllably modulates the onset of a secondary therapeutic effect associated with the at least one drug.

### BACKGROUND OF THE DISCLOSURE

Spasticity is a frequent and often significant consequence of injuries to the central nervous system, including multiple sclerosis (MS), spinal cord injury (SCI), cerebral palsy, brain trauma and stroke. People with spasticity can suffer severe, painful muscle spasms and/or rigidity during the day and night. Several types of medications have been used to treat spasticity, including: baclofen, benzodiazepines, dantrolene sodium, imidazolines, and gabapentin. Zanaflex® (Tizanidine hydrochloride, a central alpha2-adrenergic agonist) has been shown to reduce spasticity through action on the central nervous system and cause less muscle weakness than benzodiazepines or baclofen.

Tizanidine hydrochloride in the branded immediate release (IR) product, Zanaflex® is a short-acting α2 adrenergic agonist for the management of spasticity. Tizanidine is thought to reduce spasticity by blocking nerve impulses through pre-synaptic inhibition of motor neurons, which decreases spasticity without reducing muscle strength. In the multiple dose, controlled clinical studies, 48% of patients receiving any dose of tizanidine reported sedation as a secondary event. In 10% of these cases, the sedation was rated as severe compared to <1% in the placebo treated patients. The effect appears to be dose related. In a single dose study, 92% of the patients receiving 16 mg and 76% of patients receiving 8 mg reported that they had been drowsy during the 6 hour study.

Because of the short duration of effect and dose-related sedation, treatment with current tizanidine IR products (Zanaflex®) are reserved for those daily activities and times when relief of spasticity is most important. Since spasticity attacks can occur at any time of day or night, there exists an unmet medical need for a safe and efficacious once-daily treatment that controls spasticity and modulates the onset of secondary effects associated with administration of tizanidine. The present disclosure achieves these objectives, while limiting next-day side-effects, such drowsiness or dizziness, somnolence, sedation, asthenia, hypotension, and bradycardia, using a multiphasic pulsatile release pharmaceutical composition of tizanidine, or a pharmaceutically acceptable salt thereof, formulated for once-daily oral administration.

### SUMMARY OF THE DISCLOSURE

Disclosed herein, in various embodiments, are pharmaceutical compositions (e.g., dosage forms) which provide therapeutically effective blood plasma levels of a drug while controllably modulating the onset of at least one secondary therapeutic effect associated with said drug. That is, the pharmaceutical composition of the present disclosure can be formulated to provide a therapeutically effective blood plasma concentration of the drug for up to about 24 hours while taking advantage of at least one secondary therapeutic effect (e.g., sedation or somnolence) during bedtime period and reducing the at least one secondary therapeutic effect during daytime.

In embodiments, the disclosure is directed to a pharmaceutical composition comprising a skeletal muscle relaxant, wherein the composition comprises multiple skeletal muscle relaxant-containing components. In some embodiments, the components achieve a higher blood plasma concentration of the drug during bedtime, thereby providing both spasticity control and somnolence during bedtime. In further embodiments, the components achieve a lower but therapeutically effective blood plasma concentration of the drug during daytime, thereby providing spasticity control throughout the daytime but significantly reducing somnolence during daytime.

As described herein, the pharmaceutical composition exhibits specific bedtime (e.g., between about 1-10 hours after administration) and daytime (e.g., between about 8-24 after administration) drug release and pharmacokinetic profiles, following oral, “before bedtime” administration. In some embodiments, the pharmaceutical composition provides a higher plasma concentration-time profile over a first time period following administration (e.g., for about 6 to 8 hours after dosing) which provides spasticity control and induces somnolence and/or sedation, allowing for high quality sleep during the night without significantly interfering with the patient's ability to perform activities of daily life in the early morning hours. In some embodiments, the pharmaceutical composition provides lower plasma concentration-time profile over a second time period following administration (e.g., about 8 to about 24 hours after dosing) in order to minimize sedation and/or somnolence during work hours, but is sufficient to prevent spasticity attacks during daytime.

In various embodiments, the disclosure is directed to an oral pharmaceutical composition for once-daily dosing of a drug, such as tizanidine, or a pharmaceutically acceptable salt thereof. In particular embodiments, the pharmaceutical composition comprises at least three different components: an immediate release (IR) component (e.g., IR particle) comprising tizanidine, or a pharmaceutically acceptable salt thereof; a first delayed release component (e.g., timed pulsatile release (TPR) particle) comprising tizanidine or a pharmaceutically acceptable salt thereof; and a second delayed release component (e.g., TPR particle) comprising tizanidine or a pharmaceutically acceptable salt thereof. The multi-component pharmaceutical composition described herein may also be referred to as TXR or tizanidine HCl ER. In further embodiments, following oral administration, the IR component releases tizanidine or the pharmaceutically acceptable salt thereof within about 60 minutes, the first delayed release component releases tizanidine or the pharmaceutically acceptable salt thereof over a period of from about 2 hours to about 6 hours (e.g. about 4-6 hours), following a lag-time of from about 2 hours to about 6 hours (e.g. about 3-5 hours), and the second delayed release component releases of tizanidine, or the pharmaceutically acceptable salt thereof, over a period of from about 9 hours to about 12 hours (e.g., about 9-11 hours), following a lag-time of from about 7 hours to about 11 hours (e.g, about 8-10 hours).

In certain embodiments, the components are particles. In some embodiments, the particles comprise a combination of different populations of drug-containing particles for multiphasic (e.g., triphasic) pulsatile delivery of the drug. In some embodiments, the components (e.g., particles) are formulated for oral, once-daily “before bedtime” administration in patients for spasticity control for up to about 24 hours.

In certain embodiments, the first population of IR particles releases tizanidine (or a pharmaceutically acceptable salt thereof) from the IR component within about an hour following administration; the first TPR particles provide complete release of tizanidine (or a pharmaceutically acceptable salt thereof) in about 8 to about 10 hours post-dosing; and the second TPR particles, which have a lag-time of about 8 to about 11 hours, provide complete release of tizanidine (or a pharmaceutically acceptable salt thereof) in about 22 to about 24 hours post-dosing. In some embodiments, the first population of IR particles to provide bedtime blood plasma levels associated with spasticity control and sleep promotion. In some embodiments, the first TPR particles formulated to provide bedtime blood plasma levels associated with spasticity control and sleep promotion, whereas in alternative embodiments, the first TPR are formulated to provide daytime blood plasma levels that control spasticity and reduce somnolence and/or sedation In some embodiments, the second TPR particles are intended to provide daytime blood plasma levels that control spasticity and reduce somnolence and/or sedation.

In certain other embodiments, the pharmaceutical composition comprises: a “modified IR” (m-IR) component which releases tizanidine, or a pharmaceutically acceptable salt thereof, over a period of about 3-4 hours following administration; and at least one TPR component, which provides substantially complete release of tizanidine, or the pharmaceutically acceptable salt thereof, in about 8 to about 22 hours post-dosing. In some embodiments, the TPR component includes a first TPR component and a second TPR component, wherein the first TPR component provides substantially complete release of tizanidine, or the pharmaceutically acceptable salt thereof, in about 8 to about 22 hours post-dosing, and the second TPR component has a lag-time of about 8 to about 11 hours followed by substantially complete release of tizanidine, or a pharmaceutically acceptable salt thereof, in about 22 to about 24 hours post-dosing. In some embodiments, the m-IR component is intended to provide bedtime blood plasma levels associated with spasticity control and sleep promotion. In some embodiments, the TPR particles are intended to provide daytime blood plasma levels that control spasticity and reduce somnolence and/or sedation.

In some embodiments, the disclosure provides for a pharmaceutical composition comprising a therapeutically effective amount of tizanidine for once-daily dosing, wherein the pharmaceutical composition comprises: an immediate release (IR) component comprising about 10-60 wt. % of the total amount of tizanidine in the pharmaceutical composition; and a timed pulsatile release (TPR) component comprising about 20-80 wt. % of the total amount tizanidine in the pharmaceutical composition; wherein: the IR component is formulated to completely release the tizanidine within 30 minutes as measured using USP Apparatus 1 (baskets at 100 rpm) or Apparatus 2 (paddles at 50 or 75 rpm) in 700 mL of 0.1N HCl, and the TPR component is formulated to have a lag time of about 3 hours to about 8 hours and completely release the tizanidine within about 2 hr to about 8 hr after the lag time, as measured using a USP Apparatus 1 (baskets at 100 rpm) or Apparatus 2 (paddles at 50 or 75 rpm) and two-stage dissolution methodology (first 2 hr in 700 mL of 0.1N HCl at 37° C. followed by dissolution testing at pH 6.8 obtained by the addition of 200 mL of a pH modifier). In some embodiments, the pharmaceutical composition comprises from about 8 mg to about 48 mg of tizanidine, or about 12 mg to about 36 mg of tizanidine. In some embodiments, the pharmaceutical composition is formulated to provide therapeutically effective blood plasma levels of tizanidine for at least about 12 hours.

In some embodiments, the IR component is formulated to provide therapeutically effective blood plasma levels of tizanidine associated with controlling spasticity and inducing somnolence. In some embodiments, the IR component comprises from about 2 mg to about 8 mg of tizanidine. In some embodiments, the IR component is formulated to provide a first maximum blood plasma concentration (C_{max-IR}) of tizanidine within about 80%-125% of the range of from about 2.0 ng/mL to about 6.0 ng/mL following administration of about 2 mg to about 8 mg of tizanidine. In some embodiments, the IR component is formulated to provide a first maximum blood plasma concentration (C_{max-IR}) of tizanidine within about 80%-125% of the range of from about 3.0 ng/mL to about 6.0 ng/mL following administration of about 8 mg of tizanidine. In some embodiments, the IR component is formulated to provide a first maximum blood plasma concentration (C_{max-IR}) of tizanidine within about 80%-125% of the range of from about 5.0 ng/mL to about 6.0 ng/mL following administration of about 8 mg of tizanidine. In some embodiments, the IR component is formulated to provide a time to reach Cₘₐₓ (Tₘₐₓ) of tizanidine within about 80%-125% of about 1 hr to about 2 hr.

In some embodiments, the IR component is in the form of IR particles. In some embodiments, the IR particles are beads comprising an inert core and an outer surface, and the tizanidine is provided in a layer on the outer surface of the inert core. In some embodiments, the beads further comprise a polymeric binder and a solubility-enhancing polymer or an absorption-enhancing polymer, or combination thereof, which are present in an admixture with the tizanidine in the layer on the outer surface of said inert core. In some embodiments, the inert core is made of sugar or microcrystalline cellulose. In some embodiments, the IR particles are in the form of a powder blend, beads or mini-tablets comprising tizanidine and one or more pharmaceutically acceptable excipients. In some embodiments, the one or more pharmaceutically acceptable excipients are selected from the group consisting of lactose monohydrate, silicified microcrystalline cellulose, microcrystalline cellulose, pregelatinized starch, spray-dried mannitol, povidone, hypromellose, crospovidone, sodium lauryl sulfate, magnesium stearate and sodium stearyl fumarate, and combinations thereof.

In some embodiments, the beads or mini-tablets comprise a seal coating layer. In some embodiments, the seal coating comprises one or more hydrophilic polymers selected from the group consisting of hydrophilic hydroxypropylcellulose, hydroxypropyl methylcellulose, low-viscosity ethylcellulose, and mixtures thereof. In some embodiments, the hydrophilic polymer is hydroxypropyl methylcellulose.

In some embodiments, the TPR component is formulated to provide therapeutically effective blood plasma levels of tizanidine associated with controlling spasticity and reduced somnolence. In some embodiments, the TPR component comprises from about 4 mg to about 16 mg of tizanidine. In some embodiments, the TPR component is formulated to have a lag time in the range of about 3 hr to about 7 hr. In some embodiments, the TPR component is formulated to have a lag time in the range of about 3 hr to about 6 hr. In some embodiments, the TPR component is formulated to release the tizanidine within about 3 hr to about 7 hr following the lag time. In some embodiments, the TPR component is formulated to completely release the tizanidine within about 4 hr to about 6 hr following the lag time. In some embodiments, the TPR component is formulated to provide a maximum blood plasma concentration of tizanidine (C_{max, TPR-1}) of within about 80%-125% of the range of from about 2.0 ng/mL to about 6.0 ng/mL following administration of about 4 mg to about 16 mg of tizanidine. In some embodiments, the TPR component is formulated to provide a maximum blood plasma concentration of tizanidine (C_{max, TPR-1}) of within about 80%-125% of the range of from about 3.0 ng/mL to about 5.0 ng/mL following administration of about 8 mg of tizanidine. In some embodiments, the TPR component is formulated to provide a time to reach C_{max, TPR-1} (T_{max, TPR-1}) of tizanidine of between about 6 hr and about 10 hr after administration. In some embodiments, the IR component and the TPR component are formulated to provide therapeutically effective blood plasma levels of tizanidine that control spasticity for about 12-16 hours.

In some embodiments, the TPR component is in the form of particles. In some embodiments, the particles are beads or mini-tablets. In some embodiments, the TPR particles are IR particles coated with a timed pulsatile release coating. In some embodiments, the timed pulsatile release coating comprises at least one enteric polymer and at least one water-insoluble polymer. In some embodiments, the ratio of the at least one enteric polymer and the at least one water-insoluble polymer is within the range of from about 4:1 to about 1:2. In some embodiments, the ratio of the at least one enteric polymer and the at least one water-insoluble polymer is within the range of from about 3:1 to about 1:2. In some embodiments, the timed pulsatile release coating is present at about 10% to about 60% w/w based on the weight of the TPR particle. In some embodiments, the timed pulsatile release coating is present at about 20% to about 50% w/w based on the weight of the TPR particle, 25% to about 45% w/w based on the weight of the TPR particle, or about 15% to about 35% w/w based on the weight of the TPR particle. In some embodiments, the enteric polymer is selected from the group consisting of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof. In some embodiments, the enteric polymer is hydroxypropyl methylcellulose phthalate or pH-sensitive methacrylic acid/methylmethacrylate copolymers. In some embodiments, the water-insoluble polymer is selected from the group consisting of cellulose acetate, cellulose acetate butyrate, polyvinyl acetate, ethyl cellulose, pH-insensitive ethyl acrylate-methyl methacrylate copolymers, and combinations thereof. In some embodiments, the water-insoluble polymer is ethyl cellulose or a pH-insensitive ethyl acrylate-methyl methacrylate copolymer, such as ammonium methacrylate copolymers. In some embodiments, the enteric polymer is pH-sensitive methacrylic acid/methylmethacrylate copolymers and the water-insoluble polymer is an ammonium methacrylate copolymer.

In some embodiments, the TPR particles further comprise a delayed release undercoating beneath the timed pulsatile release coating. In some embodiments, the delayed release undercoating comprises an enteric polymer. In some embodiments, the enteric polymer is selected from the group consisting of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof. In some embodiments, the enteric polymer is hydroxypropyl methylcellulose phthalate or pH-sensitive methacrylic acid/methylmethacrylate copolymers. In some embodiments, the delayed release undercoating is present in an amount of from 1% to about 30% w/w based on the weight of the TPR particle.

In some embodiments, the TPR particles further comprise a barrier undercoating beneath the delayed release undercoating. In some embodiments, the barrier undercoating comprises a water insoluble polymer, a water soluble polymer, or an enteric polymer, or combinations thereof. In some embodiments, the barrier undercoating is present in an amount of from 1% to about 25% w/w based on the weight of the TPR particle.

In some embodiments, the TPR component is a first TPR component and the composition further comprises a second TPR component. In some embodiments, the second TPR component is formulated to provide therapeutically effective blood plasma levels of tizanidine associated with controlling spasticity and reduced somnolence. In some embodiments, the second TPR component comprises from about 4 to about 16 mg of tizanidine. In some embodiments, the second TPR component is formulated to have a lag time in the range of from about 6 h to about 12 h. In some embodiments, the second TPR component is formulated to have a lag time in the range of from about 8 h to about 10 h. In some embodiments, the second TPR component is formulated to release the tizanidine within about 8 h to about 14 h following the lag time. In some embodiments, the second TPR component is formulated to release the tizanidine within about 9 h to about 11 h following the lag time. In some embodiments, the second TPR component is formulated to provide a maximum blood plasma concentration of tizanidine (C_{max, TPR-2}) within about 80%-125% of the range of from about 1.5 ng/mL to about 6.0 ng/mL. In some embodiments, the second TPR component is formulated to provide a time to reach C_{max, TPR-2} (T_{max, TPR-2}) of between about 14 h and about 22 h after administration. In some embodiments, the second TPR component is formulated to provide a T_{max, TPR-2} of between about 16 h and about 20 h after administration. In some embodiments, the second TPR component is in the form of particles. In some embodiments, the TPR particles are IR particles coated with a timed pulsatile release coating. In some embodiments, the timed pulsatile release coating comprises at least one enteric polymer and at least one water-insoluble polymer. In some embodiments, the ratio of the at least one water-insoluble polymer to the at least one enteric polymer is within the range of from about 3:1 to about 1:2. In some embodiments, the timed pulsatile release coating is present on the TPR particle within the range of from about 35% to about 75% based on the weight of the coated particle. In some embodiments, the enteric polymer is selected from the group consisting of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof. In some embodiments, the enteric polymer is hydroxypropyl methylcellulose phthalate or pH-sensitive methacrylic acid/methylmethacrylate copolymers. In some embodiments, the water-insoluble polymer is selected from the group consisting of cellulose acetate, cellulose acetate butyrate, polyvinyl acetate, ethyl cellulose, pH-insensitive ethyl acrylate-methyl methacrylate copolymers, and combinations thereof. In some embodiments, the water-insoluble polymer is ethyl cellulose or a pH-insensitive ethyl acrylate-methyl methacrylate copolymer, such as an ammonium methacrylate copolymer. In some embodiments, the enteric polymer is pH-sensitive methacrylic acid/methylmethacrylate copolymers and the water-insoluble polymer is an ammonium methacrylate copolymer.

In some embodiments, the second TPR particles comprise TPR particles disclosed above coated with a barrier coating applied over the timed pulsatile release coating. In some embodiments, the barrier coating comprises at least one water-insoluble polymer and at least one water-soluble polymer. In some embodiments, the water-insoluble polymer is selected from the group consisting of cellulose acetate, cellulose acetate butyrate, polyvinyl acetate, ethyl cellulose, pH-insensitive ethyl acrylate-methyl methacrylate copolymers, and mixtures thereof. In some embodiments, the water-soluble polymer is selected from a low molecular weight polyethylene glycol and hydroxypropyl methyl cellulose.

In some embodiments, the tizanidine is in the form of a hydrochloride salt. In some embodiments, the pharmaceutical composition is a capsule.

The pharmaceutical composition of the present disclosure may be used to treat any neurological disease or condition for which skeletal muscle relaxants are administered. In embodiments, the pharmaceutical compositions may be used to treat spasticity, multiple sclerosis, amyotrophic lateral sclerosis, cerebral palsy, stroke, brain injury, or spinal cord injury.

Also provided herein are methods of (a) treating spasticity in a patient having a neurological disease; (b) improving sleep or sleep quality in a patient with a neurological disease; (c) reducing daytime fatigue or sleepiness in a patient having a neurological disease and being treated with skeletal muscle relaxants; and/or (d) improving daytime quality of life in a patient having a neurological disease and being treated with skeletal muscle relaxants, said methods comprising administering a pharmaceutical composition disclosed herein.

In some embodiments, the method of treating spasticity in a patient in need thereof comprises administering the pharmaceutical compositions disclosed herein. In some embodiments, the method achieves at least one of the following: (i) ameliorates spasticity; (ii) improves sleep or sleep quality; (iii) reduces daytime fatigue or sleepiness; and (iv) improves daytime quality of life. In some embodiments, the patient has a neurological disease. In some embodiments, he neurological disease is selected from cerebral palsy, multiple sclerosis, stroke, restless leg syndrome, spinal cord injury, and traumatic brain injury.

In some embodiments, the present disclosure provides for a method of treating spasticity and modulating the onset of somnolence over about a 24 hour period in a patient in need thereof, comprising administering, once daily, the pharmaceutical compositions described herein. In some embodiments, the pharmaceutical composition is administered to a patient at bedtime. In some embodiments, the average blood plasma concentration of tizanidine during the first about 8-10 hours following administration induces somnolence and provides spasticity control. In some embodiments, the average blood plasma concentration of tizanidine is within about 80% to about 125% of the range of from about 2.0 ng/mL to about 5.5 ng/mL during the first about 8-10 hours following administration. In some embodiments, the average blood plasma concentration of tizanidine during a period of about 8 to about 24 hours following administration provides spasticity control and reduced somnolence. In some embodiments, the average blood plasma concentration of tizanidine is within about 80% to about 125% of the range of from about 1.0 ng/mL and about 4.0 ng/mL for about 12 to about 22 hours following administration.

In some embodiments, the disclosure provides pharmaceutical compositions for once daily dosing for use in treating spasticity in a patient in need thereof. In some embodiments, the disclosure provides for tizanidine in a medicament for use in method to treat spasticity in a patient in need thereof, wherein the tizanidine is administered in a pharmaceutical composition described herein. In some embodiments, the pharmaceutical composition is administered before bedtime.

### BRIEF DESCRIPTIONS OF THE FIGURES

FIG. 1 shows the simulated tizanidine concentration in plasma in healthy subjects. Also plotted are observed data from Shah et al., (2006); Shellenberger et al., (1999); and Tse et al., (1987). Note: the observed data are normalized to 8 mg based on dose proportionality.

FIG. 2 shows simulated PK profiles for IR, TPR2, TPR3, TPR(2+3), and target (Ideal) PK profiles at IR:TPR2:TPR3 Dose Ratio of 8 mg:14 mg:10 mg (Total dose: 32 mg).

FIG. 3 shows simulated PK profiles for component populations of IR, TPR1, TPR2 particles at a dose ratio of 17:28:55 of once-daily TXR delivery system.

FIG. 4 shows the in vitro release profiles for once-daily TXR delivery systems (TXR-1, TXR-2, and TXR-3) with a total daily dose of 24 mg.

FIG. 5A shows the simulated PK profiles for once-daily TXR delivery systems (TXR-1, TXR-2, and TXR-3) with a total dose of 24 mg. FIG. 5B shows the simulated PK profile for once-daily TXR-4 comprising populations of IR, TPR-1 and TPR-2 at a ratio of 17% (4.8 mg):27% (7.6 mg):56% (15.6 mg) with a total dose of 28 mg.

FIG. 6A shows the in vitro release profiles for tizanidine TPR bead populations comprising IR beads coated with EC-10/HP 55/TEC at 45/40/15, 55/30/15, or 60/25/15 for a weight gain of 15%. FIG. 6B shows the in vitro release profiles for tizanidine TPR bead populations comprising IR beads coated with EC-10/HP 55/TEC at 45/40/15, 55/30/15, or 60/25/15 for a weight gain of 30%. FIG. 6C shows the in vitro release profiles for tizanidine TPR bead populations comprising IR beads coated with EC-10/HP 55/TEC at 45/40/15, 55/30/15, or 60/25/15 for a weight gain of and 45%.

FIG. 7 shows the in vitro release profiles for Tizanidine TPR bead populations comprising IR beads first coated with a barrier coating with HP 55/TEC at 90/10 for a weight gain of 20% followed by a TPR coating with EC-10/HP 55/TEC at 55/30/15 for a weight gain of 20%, 40%, 50%, 55% or 60%.

FIG. 8 shows the in vitro release profiles for Tizanidine TPR bead populations comprising IR beads coated with a TPR coating with EUDRGIT RSPO/L100/S100 at 50/25/25 for a weight gain of 12%, 24%, 30%, 36%, 40% or 45%

FIG. 9A shows the in vitro release profiles for tizanidine XR prototypes Prototype 1, 2, and 3 with compositions listed in the table below. Specifically, the TPR-1 beads comprising IR beads coated with RSPO/L100/S100/TEC/Talc 50/25/25 for a weight gain of 25% or 30%, and TPR-2 beads comprising IR beads coated with RSPO/L100/S100/TEC/Talc 50/25/25 for a weight gain of 40% were used. FIG. 9B shows the simulated PK profiles for prototypes 1, 2, and 3.

| Prototype | IR/mIR* | TPR-1 | TPR-2 |
|---|---|---|---|
| 1: (20%:30%:50%) | IR_{0.5 h} | Batch 17- TPR | Batch 17- TPR |
| | | Coat at 25% | Coat at 40% |
| 2: (20%:30%:50%) | mIR_{3-4 h} | Batch 17- TPR | Batch 17- TPR |
| | | Coat at 25% | Coat at 40% |
| 3: (20%:30%:50%) | mIR_{3-4 h} | Batch 17- TPR | Batch 17- TPR |
| | | Coat at 30% | Coat at 40% |

*Duration for complete Tizanidine release is about 30 minutes or 3 to 4 hours

### DETAILED DESCRIPTION OF THE DISCLOSURE

The following description includes information that may be useful in understanding the present disclosure. It is not an admission that any of the information provided herein is prior art or that any publication specifically or implicitly referenced is prior art.

All documents cited herein are incorporated by reference in their entirety for all purposes to the same extent as if each individual document was specifically and individually indicated to be incorporated by reference.

As used in this specification and appended claims, the singular forms “a”, “an”, and “the” include plural referents unless the content clearly dictates otherwise. Thus, for example, reference to “a water-soluble polymer” includes a mixture of one or more water-soluble polymers, and the like.

The term “condition” refers to any illness or disability, requiring medication and/or medical attention.

The terms “drug”, “active”, or “active ingredient” as used herein refers to a central-acting α2 adrenoceptor agonist, in particular tizanidine, including any polymorphic form, the corresponding deuterated analog, and pharmaceutically acceptable salts thereof. Thus, any reference to a drug (e.g., tizanidine) herein also includes the pharmaceutically acceptable salts thereof.

The term “particles”, as used herein, includes beads, granules, pellets, micro-particles, micro-particulates, mini-tablets, or a powder mix or blend (e.g. suitable for preparing mini-tablets). The term “drug micro-particulates” or “drug micro-particles” refer to drug particles with particle size in the micrometer range.

The term “multiphasic”, as used herein, means a pharmaceutical composition having two or more pulsatile phases each of which provide plasma tizanidine concentration-time profiles.

The term “triphasic”, as used herein, refers to a composition having three pulsatile phases each of which provide plasma tizanidine concentration-time profiles. For example, in some embodiments, one pulsatile component is a rapid release pulse (e.g. immediate release pulse) which occurs within about 60 minutes (e.g., within about 30 minutes), and the second and third pulsatile components each have a pseudo-zero order release profile over several hours (e.g. from about 2 hours to about 10 hours and from about 10 hours to about 22 hours, respectively) following respective lag-times. Alternatively, the first pulsatile component may be a modified immediate release pulse which occurs over the first 3-4 hour period post-dosing.

The term “patient”, as used herein, means a subject who has presented a clinical manifestation of a particular disorder with symptom or symptoms suggesting the need for treatment, who is treated preventatively or prophylactically for a condition, or who has been diagnosed with a condition to be treated.

The term “disposed over”, as used herein, refers to a coating over a substrate, that denotes a relative location of the coating, e.g. a coating disposed over a substrate requires that the coating is outside of the substrate but need not be in direct contact with the substrate, i.e. another coating or material could be interposed there between. For example, a first coating “disposed over” a substrate can be in direct contact with the substrate, or one or more intervening materials or coatings can be interposed between the first coating and the substrate.

The term “immediate release” or “IR” refers to a particle, a component, or a pharmaceutical composition (such as a capsule or an orally disintegrating tablet, a hot melt extruded or spray-congealed composition) wherein at least about 90% (e.g. about 91% or greater, about 92% or greater, about 93% or greater, about 94% or greater, about 95% or greater, about 96% or greater, about 97% or greater, about 98% or greater, about 99% or greater, or about 100%) of tizanidine is released within about 30 minutes, following oral administration or when in vitro dissolution tested per United States Pharmacopeia using USP Apparatus 1 (baskets at 100 rpm) or Apparatus 2 (paddles at 50 or 75 rpm) in 700 mL of 0.1N HCl at 37° C. for 2 hours followed by adjusting to pH 6.8 upon addition of 200 mL of a pH modifier.

The term “modified IR” or “m-IR” refers to particles, a component or a pharmaceutical composition (such as a capsule or an orally disintegrating tablet, a hot melt extruded or spray-congealed dosage form), wherein at least about 50%, or at least about 75%, or at least about 90% (e.g. about 95% or greater) of tizanidine is released within about 3-6 hours following oral administration or when in vitro dissolution tested per United States Pharmacopeia using USP Apparatus 1 (baskets at 100 rpm) or Apparatus 2 (paddles at 50 or 75 rpm) in 700 mL of 0.1N HCl at 37° C. for 2 hours. In some embodiments, the m-IR component provides for an initial rapid release (e.g., within about 1 hour) followed by complete drug release from the modified IR component within a time period of about 3-6 hours (e.g., about 3-4 hours) post-dosing.

The term “timed pulsatile release” or “TPR” refers to a particle, a component, or a pharmaceutical composition (such as a capsule or an orally disintegrating tablet, a hot melt extruded or spray-congealed composition) having a timed, pulsatile release profile, i.e., a time-controlled release occurring several hours after oral administration (i.e., after a lag-time). The term “delayed release” encompasses “timed pulsatile release”. In some embodiments, the timed pulsatile release profile is characterized by a single pulse or series of pulses occurring several hours after oral administration (i.e., after a lag-time).

The term “lag time”, as used herein, refers to the amount of time that lapses following administration before an amount of the drug (e.g., >about 10%) begins to be released from the pharmaceutical composition or component thereof. That is, during the lag time, no more than about 10% of the drug is released.

The term “complete drug release,” “complete release,” and variants thereof, as used herein, refers release of least about 90% (e.g., about 91%, about 92%, about 93%, about 94%, about 95%, about 96%, about 97%, about 98%, about 99%, or about 100%) of the amount of the drug from the pharmaceutical composition of component thereof. For example, when release occurs over a period of about 4 hours, at least about 90% of the drug is release within 4 hours.

The term “pH-sensitive,” as used herein, refers to a polymer which responds to the changes in the pH of the surrounding medium by undergoing conformational changes. Such polymers may swell, collapse, and/or change (e.g., protonate or deprotonate) depending on the pH of their environment. For example, a pH-sensitive polymer may be stable in the acidic pH of the stomach, but swells/collapses at higher pH conditions in the upper intestinal tract below the stomach. Such pH-sensitive polymers are commonly referred to as enteric polymers.

The term “pH-insensitive,” as used herein, refers to a polymer which does not respond to the changes in the pH of the surrounding medium by undergoing conformational changes.

The terms “plasma concentration” and “elimination half-life” have their generally accepted meanings as defined in the FDA Guidance for Industry: Bioavailability and Bioequivalence Studies for Orally Administered Drug Products—General Considerations (issued March 2003).

The term “somnolence” refers to a feeling of drowsiness or sleepiness which is associated with the administration of tizanidine or a pharmaceutically acceptable salt thereof. The terms “somnolence”, “drowsiness”, “sleepiness”, and equivalents thereof, are used interchangeably herein.

The term “sedation” as used herein refers to the depression of a patient's awareness to the environment and reduction of his or her responsiveness to external stimulation. It also refers to the act of facilitating faster onset of sleep by a sedative drug.

As used herein, the term “bedtime” means the time at which an individual intends to retire to sleep and continues until said individual awakes from a full night's sleep, which is commonly about hours. The term “before bedtime” refers to the period of time before retiring to sleep. Thus, typically the term “before bedtime” refers to a period of time within about 1 hour before retiring to sleep, e.g., within about 30 minutes before retiring to sleep, or within about 15 minutes before retiring to sleep. In particular embodiments, the term “before bedtime” includes a time period of from about 5 minutes to about 30 minutes before retiring to sleep.

The term “about” is used herein to refer to a numerical quantity, and includes “exactly.” For example, “about 1 hour” includes 1 hour, exactly, as well as values close to 1 hour (e.g., 50 minutes, 55 minutes, 59 minutes, 61 minutes, 65 minutes, 70 minutes, etc.).

Unless indicated otherwise, all percentages and ratios are calculated by weight based on the total component, such as coated particle, or composition, such as capsule or tablet.

Unless stated otherwise, the amount of coatings or layers described herein (the “coating weight”) is expressed as the percentage weight gain provided by the coating, relative to the initial weight of the particles or beads prior to coating. Thus, a 10% coating weight refers to a coating which increases the weight of a particle or bead by 10%.

Provided herein, in various embodiments, are pharmaceutical compositions comprising skeletal muscle relaxants for once-daily dosing, such as, but not limited to, cyclobenzaprine, dantrolene sodium, methocarbamol, metaxalone, carisoprodol, diazepam, benzodiazepines, baclofen, onabotulinumtoxin A, methocarbamol, metaxalone, carisoprodol, and tizanidine, and pharmaceutically acceptable salts or derivatives thereof. In particular embodiments, the pharmaceutical compositions comprise tizanidine or a pharmaceutically acceptable salt thereof. Any reference to skeletal muscle relaxants, such as, tizanidine in the present disclosure also includes any of its pharmaceutically acceptable salts. Accordingly, the present disclosure uses “tizanidine” and “tizanidine, or a pharmaceutically acceptable salt thereof” interchangeably, and any reference to “tizanidine” necessarily includes its pharmaceutically acceptable salts, such as tizanidine HCl.

Tizanidine is a central alpha-2-adrenergic agonist indicated for the management of spasticity. Tizanidine has a molecular formula of C₉H₈ClN₅S and molecular weight of 253.02 Tizanidine is commonly administered in its salt form. A non-limiting example of a pharmaceutically acceptable salt of tizanidine is tizanidine HCl with a molecular formula of C₉H₈ClN₅S.HCl and molecular weight is 290.2 and its structure is shown below:

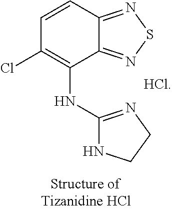

Tizanidine is as effective as other antispasmodic drugs and has a better tolerability profile compared with baclofen and diazepam; however, patients on tizanidine typically experience secondary effects, such as dry mouth, somnolence, sedation, drowsiness, dizziness, asthenia, hypotension, and bradycardia, after administration. While some of these secondary effects—somnolence and sedation—also provide a therapeutic benefit, such as sleep promotion and improving the quality sleep, such secondary effects are not desired during daytime because it interferes with the patient's ability to perform daily activities. Accordingly, the pharmaceutical compositions disclosed herein can modulate the onset of at least one of these secondary effects to provide a therapeutic benefit to the patient during bedtime, but not daytime, while maintaining spasticity control for about 12-24 hours (e.g., about 12, about 13, about 14, about 15, about 16, about 17, about 18, about 19, about 20, about 21, about 22, about 23, about 24, inclusive of all values and subranges therebetween).

In various embodiments, the present compositions control spasticity and modulate the onset of somnolence and/or sedation by providing higher blood plasma concentrations of tizanidine at bedtime. In various embodiments, the present compositions control spasticity and reduce somnolence and/or sedation in the daytime by providing comparatively lower therapeutically effective blood plasma concentrations of tizanidine during the daytime. The tests disclosed in Example 7 (KSS, CANTAB Reaction Time Test, and Bond-Lader VAS for Mood and Alertness) can be used to determine whether certain doses or blood plasma levels induce somnolence and/or sedation. In some embodiments, reduced somnolence and/or sedation refers to a reduction of such events (e.g., in a population of patients) by at least about 10% (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%) compared to the number of events of somnolence and/or sedation reported for an equivalent dose of tizanidine in an immediate release formulation. For example, a TPR component of the present disclosure having about 16 mg of tizanidine may reduce somnolence and/or sedation by at least about 10% compared to an immediate release formulation having about 16 mg of tizanidine. Such a reduction may be achieved by lowering the Cmax and/or smoothing the blood plasma-time concentration profile.

In various embodiments, the pharmaceutical compositions disclosed herein comprise multiple components. In other embodiments, a portion of the components are formulated to provide higher average blood plasma concentrations of the drug during bedtime, which provide spasticity control and induce somnolence and/or sedation during bedtime. In some embodiments, a portion of the components are formulated to provide comparatively lower average blood plasma concentrations of the drug during daytime, which provide spasticity control but reduce somnolence and/or sedation during daytime.

In order to achieve such therapeutic effects, the present compositions are formulated to release tizanidine in a series of pulses. A first pulse is intended to control spasticity and induce somnolence and/or sedation at bedtime, when such therapeutic effects are most important to, and desired by, the patient and do not interfere with daily activities. Due to the short half-life of tizanidine, plasma levels dissipate and muscle spasms return in the morning hours, which can prevent a patient from getting out of bed and starting his/her daily activities. To address this, a second pulse is intended to coincide with the time in which the patient wakes up to begin his/her daily activities and provides the necessary spasticity control in the morning hours. However, certain blood plasma levels of tizanidine (e.g., those provided by immediate release 8-16 mg doses) cause somnolence and/or sedation, which also interferes with the patients' ability to get out of bed in the morning. To address this, the second pulse is released over an extended period of time which flattens the blood plasma concentration curve compared to an immediate release composition, such that the therapeutic effect is maintained but somnolence and/or sedation is reduced. Similar to the second pulse, the third pulse (when present) is released over an extended period during the daytime to provide spasticity control and minimizing somnolence and/or sedation.

In some embodiments, the pharmaceutical compositions of the disclosure (TXR, e.g. from about 12-36 mg once-daily capsule) can be formulated to provide a higher average blood plasma concentration profile of Tizanidine (e.g., within about 80% to about 125% of the range of from about 2.5-5.5 ng/mL) during bedtime in order to control spasticity and induce sedation and/or somnolence. In further embodiments, the pharmaceutical composition may be designed to provide a lower average blood plasma concentration profile of Tizanidine (e.g., about 1.0-4.0 ng/mL) during daytime to control spasticity and reduce secondary effects such as sedation and/or somnolence until the next dosing.

In some embodiments, the components include an immediate release (IR) component and at least one delayed release component (e.g., 2 delayed release components). In other embodiments, the at least one delayed release component is at least one timed pulsatile release (TPR) component. In still other embodiments, the pharmaceutical compositions disclosed herein include two TPR components. In some embodiments, the components are particles. In some embodiments, the particles are a combination of different populations of drug-containing particles for multiphasic (e.g., biphasic or triphasic) pulsatile delivery of the drug. Accordingly, in some embodiments, the pharmaceutical composition comprises a population of IR particles, a first population TPR particles (TPR-1), and a second population of TPR particles (TPR-2). In some embodiments, the components (e.g., particles) are formulated into a dosage form for oral, once-daily “before bedtime” administration in patients for spasticity control for about 24 hours.

### Compositions and Dosage Forms

In some embodiments, the pharmaceutical compositions described herein comprise a total amount of tizanidine (or a pharmaceutically acceptable salt thereof) in the range of from about 1 mg to about 100 mg, e.g., from about 12 mg to about 48 mg, including about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, and about 48 mg, inclusive of all values and subranges therebetween. In some embodiments, the pharmaceutical compositions comprise from about 8-48 mg of tizanidine or a pharmaceutically acceptable salt thereof. In some embodiments, the pharmaceutical compositions comprise from about 12-36 mg of tizanidine or a pharmaceutically acceptable salt thereof. In particular embodiments, the pharmaceutical compositions comprise about 24 mg or about 28 mg of tizanidine or a pharmaceutically acceptable salt thereof.

The pharmaceutical compositions disclosed herein include multiple drug-containing components, and the present disclosure contemplates that the total amount of tizanidine can be divided among the various drug-containing components in any manner. Further, because patients have different needs (e.g., depending on the condition treated and severity) and there may be variations in response to particular doses within patient populations, the present disclosure envisions that that the dose of tizanidine in each of the components can be adjusted to suit an individual patient's needs. For example, an IR component containing 8 mg of tizanidine may be suitable to induce somnolence in some patients, whereas other patients may need 6 mg or 10 mg.

Each of the drug component components may include from about 10 wt % to about 90 wt % of the total dose of tizanidine in the pharmaceutical composition. For example, in some embodiments, the instant release component may comprise from about 10 wt % to about 90 wt % of the total dose of tizanidine, e.g., about 15%, about 20%, about 25%, about 30%, about 35%, about 40%, about 45%, about 50%, about 55%, about 60%, about 65%, about 75%, about 80%, about 85%, and about 90%, inclusive of all values and subranges therebetween. In some embodiments, the modified instant release component may comprise from about 10 wt % to about 90 wt % of the total dose of tizanidine, e.g., about 15%, about 20%, about 25%, about 30%, about 35%, about 40%, about 45%, about 50%, about 55%, about 60%, about 65%, about 75%, about 80%, about 85%, and about 90%, inclusive of all values and subranges therebetween. In some embodiments, the delayed release component may comprise from about 10 wt % to about 90 wt % of the total dose of tizanidine, e.g., about 15%, about 20%, about 25%, about 30%, about 35%, about 40%, about 45%, about 50%, about 55%, about 60%, about 65%, about 75%, about 80%, about 85%, and about 90%, inclusive of all values and subranges therebetween. In some embodiments, a timed pulsatile release component may comprise from about 10 wt % to about 90 wt % of the total dose of tizanidine, e.g., about 15%, about 20%, about 25%, about 30%, about 35%, about 40%, about 45%, about 50%, about 55%, about 60%, about 65%, about 75%, about 80%, about 85%, and about 90%, inclusive of all values and subranges therebetween. In some embodiments the timed pulsatile release component comprises a first timed pulsatile release component and a second timed pulsatile release component; the first timed pulsatile release component may comprise from about 10 wt % to about 90 wt % of the total dose of tizanidine, e.g., about 15%, about 20%, about 25%, about 30%, about 35%, about 40%, about 45%, about 50%, about 55%, about 60%, about 65%, about 75%, about 80%, about 85%, and about 90%, inclusive of all values and subranges therebetween; and the second timed pulsatile release component may comprise from about 10 wt % to about 90 wt % of the total dose of tizanidine, e.g., about 15%, about 20%, about 25%, about 30%, about 35%, about 40%, about 45%, about 50%, about 55%, about 60%, about 65%, about 75%, about 80%, about 85%, and about 90%, inclusive of all values and subranges therebetween. The present disclosure contemplates all combinations of the above wt % of tizanidine may be used in the present compositions. As used herein, the “wt %” of tizanidine refers to the amount of tizanidine in a particular component of the pharmaceutical composition relative to the total amount of tizanidine in the pharmaceutical composition. Because the amount of tizanidine in each of the components may vary, the total wt % of tizanidine in the components may be expressed in ranges. It will be appreciated that the sum of the wt % of tizanidine the composition does not exceed 100%, even though, e.g., the sum of the maximum values in two or more ranges may exceed 100%.

In some embodiments, the pharmaceutical compositions described herein comprise: (i) an immediate release component comprising about 10-40 wt. % of the total dose of tizanidine or a pharmaceutically acceptable salt thereof; (ii) a first timed pulsatile release component comprising about 25-50 wt. % of the total dose of tizanidine or a pharmaceutically acceptable salt thereof; and (iii) a second timed pulsatile release component comprising about 30-60 wt. % of the total dose of tizanidine or a pharmaceutically acceptable salt thereof.

In some embodiments, the weight ratio of tizanidine (or the pharmaceutically acceptable salt thereof) in the immediate release component (IR):the first timed pulsatile release component (TPR1):the second timed pulsatile release component (TPR2) is in the range of from about 2 to about 16 (IR):about 2 to about 16 (TPR1):about 8 to about 16 (TPR2), respectively. In some embodiments, the weight ratio of tizanidine or the pharmaceutically acceptable salt thereof is in the range of from about 2 to about 8 (IR):about 10 to about 14 (TPR1):about 9 to about 19 (TPR2), respectively. In some embodiments, the weight ratio of tizanidine or the pharmaceutically acceptable salt thereof is about 6 to about 10 (IR):about 6 to about 10 (TPR1):about 10 to about 16 (TPR2), respectively. In particular embodiments, the weight ratio of tizanidine or the pharmaceutically acceptable salt thereof is about 8:about 8:about 16, respectively. In other particular embodiments, the weight ratio of tizanidine or the pharmaceutically acceptable salt thereof is about 8:about 8:about 8, respectively.

In some embodiments, the pharmaceutical compositions described herein comprise: (i) an immediate release component comprising about 10-60 wt. % of the total dose of tizanidine or a pharmaceutically acceptable salt thereof; (ii) a timed pulsatile release component comprising about 20-80 wt. % of the total dose of tizanidine or a pharmaceutically acceptable salt thereof. In some embodiments, the time pulsatile release component comprises a first timed pulsatile release component and second timed pulsatile release component, wherein the first timed pulsatile release component comprises about 20-50 wt. % of the total dose of tizanidine or a pharmaceutically acceptable salt thereof, and the second timed pulsatile release component comprises about 20-50 wt. % of the total dose of tizanidine or a pharmaceutically acceptable salt thereof

In some embodiments, the weight ratio of tizanidine (or the pharmaceutically acceptable salt thereof) in the immediate release component (IR):the timed pulsatile release component (TPR) is in the range of from about 2 to about 16 (IR):and about 4 to about 16 (TPR), respectively. In other embodiments, the weight ratio of tizanidine or the pharmaceutically acceptable salt thereof is about 6 to about 10 (IR):about 8 to about 26 (TPR), respectively. In some embodiments, the weight ratio of tizanidine or the pharmaceutically acceptable salt thereof in the immediate release component to the timed pulsatile release component, is in the range of from about 1:6 to about 2:1, including about 1:6, about 1:5, about 1:4, about 1:3, about 1:2, about 1:1 and about 1:2.

In some embodiments, the pharmaceutical compositions described herein comprise: (i) a modified immediate release component comprising about 20-80 wt. % of the total dose of tizanidine or a pharmaceutically acceptable salt thereof; (ii) at least one timed pulsatile release component comprising about 20-60 wt. % of the total dose of tizanidine or a pharmaceutically acceptable salt thereof. In some embodiments, the at least one times pulsatile release component comprises a first and second timed pulsatile release component. In some such embodiments, the first timed pulsatile release component comprises about 20-50 wt. % of the total dose of tizanidine or a pharmaceutically acceptable salt thereof, the second timed pulsatile release component comprising about 20-50 wt. % of the total dose of tizanidine or a pharmaceutically acceptable salt thereof.

In some embodiments, the weight ratio of tizanidine or the pharmaceutically acceptable salt thereof in the modified immediate release component to the at least one timed pulsatile release component, and the first timed pulsatile release component is in the range of from about 5 to about 8:about 9 to about 19, respectively.

The compositions of the present disclosure may prepared using well known pharmaceutical principles, including, for example, utilizing general methods described in U.S. Pat. Nos. 6,627,223; 6,663,888; 6,500,454; 6,663,888; 9,161,918; and 9,161,919, each of which are hereby incorporated by reference in its entirety.

The present disclosure envisions that each of components of the disclosed pharmaceutical compositions may be formulated in a variety of forms. In some embodiments, the IR component can be disposed over a TPR component, or the IR component can be distinct from the TPR component. For example, an IR component can be in the form of a coating that can be disposed over a TPR capsule or matrix, or IR particle can be combined with TPR particles in a capsule or tablet. In other embodiments, the IR component can be provided as a layer of a bi- or multi-layer tablet.

In a particular embodiment, compositions of the disclosure comprise multiple populations of particles. The particles may be in any suitable form, e.g., granules, granulated-extruded-spheronized pellets, DIFFUCAPS° beads, mini-tablets, or microparticles, optionally coated with one or more functional polymers to provide the desired drug release profiles. In other embodiments, the particle is the solid carrier with a drug deposited on the solid carrier by methods known in the art, e.g. fluidized bed coating or pan coating methods. In some embodiments, the particle may be an inert bead or an inert particle or sphere. In other embodiments, the particle may be a non-pareil seed, an acidic buffer crystal, an alkaline buffer crystal, or an encapsulated buffer crystal.

In some embodiments, the particle may be a sugar sphere, cellulose sphere, lactose sphere, lactose-microcrystalline (MCC) sphere, mannitol-MCC sphere, or silicon dioxide sphere. In other embodiments, the particle may be a saccharide, a sugar alcohol, or combinations thereof. Suitable saccharides include lactose, sucrose, maltose, and combinations thereof. Suitable sugar alcohols include mannitol, sorbitol, xylitol, maltitol, arabitol, ribitol, dulcitol, iditol, isomalt, lactitol, erythritol and combinations thereof.

In particular embodiments, the IR component (such as particle) is a granule, granulated-extruded-spheronized pellet, DIFFUCAPS®, bead or mini-tablet comprising tizanidine or a pharmaceutically acceptable salt thereof. IR particles may be prepared by coating inert cores or inert spheres (e.g. sugar spheres or non-pareil seeds, cellulosic spheres or microcrystalline cellulose spheres) with tizanidine or a pharmaceutically acceptable salt and a polymeric binder (eg., polyvinyl pyrrolidone, Povidone or PVP) in solution using a fluid bed coater, such Glatt GPCG 1, GPCG 3 or GPCG 5, (pilot scale) or Glatt GPCG 120 (commercial scale). The immediate release particles may further be coated with a seal coat material (such as OPADRY® CLEAR containing hydrophilic polymer, low viscosity Hypromellose or HPMC E5).

The particle may also comprise a solubility or absorption-enhancing polymer admixed with the drug and disposed over the outer surface of said inert core or sphere. A “solubility enhancing polymer” refers to a water-soluble polymer capable, at suitable concentrations, of improving the solubility of the drug at physiologically relevant conditions. Non-limiting examples of suitable solubility-enhancing polymers include polyvinylpyrrolidone, copolymers of vinyl acetate/vinylpyrrolidone, methylcellulose, hydroxypropyl cellulose, hydroxypropyl methylcellulose acetate succinate, polyethylene oxide, polyethylene glycol, and cyclodextrin. An “absorption-enhancing polymer” refers to a polymer capable, at suitable concentrations, of improving absorption of the drug after administration. Non-limiting examples of suitable absorption-enhancing polymers include cellulose acetate phthalate, methyl cellulose acetate phthalate, ethyl cellulose acetate phthalate, hydroxypropyl cellulose acetate phthalate, hydroxypropyl methylcellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.. In particular embodiments, the absorption-enhancing polymer is polyvinyl acetate-co-vinyl pyrrolidone at 60:40 (KOLLIDON® VA 64); ethylene glycol/vinyl caprolactam/vinyl acetate copolymer at 13:57:30 (SOLUPLUS®; cationic copolymer based on dimethylaminoethyl methacrylate, butyl methacrylate, and methyl methacrylate (EUDRAGIT® EPO); polyethylene oxide; polyethylene glycol; hydroxypropyl cellulose; hypromellose; hypromellose phthalate (HP-50); polyvinyl pyrrolidone (PVP, povidone); d-alpha-tocopheryl polyethylene glycol 1000 succinate (TPGS); lipids; triglycerides; and bile acids such as sodium cholate and mixtures thereof.

In embodiments, the delayed release component (e.g., TPR component) can employ any appropriate technology which delays the release of the drug after administration. Non-limiting examples of suitable technologies include diffusion systems (e.g., reservoir devices and matrix devices) in which drug release is controlled by the rate of drug diffusion through a polymer, dissolution systems in which drug release is controlled by the rate at which the system (e.g., polymer coating, matrix, etc.) dissolves in biological fluids, osmotic pumps, ion-exchange resins, and the like. For example, various matrices with different diffusions rates (e.g., having different mixtures of polymers) can be combined to achieve the pharmacokinetic characteristics described herein. Similarly, osmotic pumps having different pulse rates can be combined to achieve the pharmacokinetic characteristics described herein.

In some embodiments, the delayed release component is a TPR component. In some embodiments, the TPR component may be a drug containing IR component (e.g. IR bead, mini-tablet, etc.) formulated with one or more functional polymers. Functional polymers may be used for coating particles or incorporated into polymer matrix-containing granules to modify the properties of the particles, e.g. lag time and release rate. The term “functional polymer” means a polymer comprising a water-insoluble polymer, a water-soluble polymer, or an enteric polymer, or a mixture thereof, and optionally including a plasticizer, anti-taking agent, and the like. In embodiments in which the pharmaceutical compositions disclosed herein (or components thereof) include a coating comprising two or more functional polymers, the polymers can be applied as distinct layers, or as a single layer which contains a mixture of the two or more functional polymers.

Non-limiting examples of water-soluble polymers include hydroxypropyl methylcellulose (aka HPMC or hypromellose), hydroxypropylcellulose, polyvinylpyrrolidone, and polyethylene glycol (e.g., low molecular weight polyethylene glycol, such as those having a molecular weight less than about 10,000 g/mol, less than about 9,000 g/mol, less than about 8,000 g/mol, less than about 7,000 g/mol, less than about 6,000 g/mol, less than about 5,000 g/mol, less than about 4,000 g/mol, or less than about 3,000 g/mol).

Non-limiting examples of water-insoluble polymers include ethylcellulose, cellulose acetate, cellulose acetate butyrate, polyvinyl acetate, pH-insensitive copolymers of ethyl acrylate and methyl methacrylate optionally copolymerized with a trialkyl ammonioalkyl methacrylate (e.g., ammonium methacrylate copolymers), such as EUDRAGIT® RL (e.g. EUDRAGIT® RLPO, RL100, RL12,5, and RL30D), EUDRAGIT® RS (e.g. EUDRAGIT® RSPO, RS100, RS12,5, and RS30D), EUDRAGIT® NE (e.g. EUDRAGIT® NE30D and NE40D), and mixtures thereof. In a particular embodiment, the water-insoluble polymer comprises ethylcellulose with a mean viscosity of 10 cps in a 5% solution in 80/20 toluene/alcohol measured at 25° C. on an Ubbelohde viscometer.

Non-limiting examples of enteric polymers include cellulose acetate phthalate, hydroxypropyl methylcellulose phthalate (HP-55), hydroxypropyl methylcellulose acetate succinate (MG, LG, or HG dissolving above a pH of 5.0, 5.5 or 6.5, respectively), polyvinyl acetate phthalate, pH-sensitive methacrylic acid/methylmethacrylate copolymers (e.g., EUDRAGIT® L (e.g., L100, L100-55, and L12,5:), EUDRAGIT® S (e.g. S100 and S12,5), and EUDRAGIT® FS), shellac, and mixtures thereof. These enteric polymers may be used as a solution in a solvent mixture or an aqueous dispersion. Commercially available materials that may be used include methacrylic acid/methylmethacrylate copolymers sold under the trademark EUDRAGIT® (L100, S100, L30D, FS) from Evonik, Cellacefate® (cellulose acetate phthalate) from Eastman Chemical Co., AQUATERIC® (cellulose acetate phthalate aqueous dispersion) from FMC Corp., AQOAT® (hydroxypropyl methylcellulose acetate succinate aqueous dispersion) from Shin Etsu K.K®, and AQUASOLVE AS™ (HPMCAS-LG/MG/HG commercially available from Ashland Specialty Ingredients).

In embodiments, the water-insoluble or enteric polymer may include a plasticizer. Non-limiting examples of plasticizers include glycerin, triacetin, citrate esters, triethyl citrate, acetyltriethyl citrate, tributyl citrate, acetyl tri-n-butyl citrate, diethyl phthalate, dibutyl sebacate, substituted triglycerides and glycerides, monoacetylated and diacetylated glycerides (e.g., Myvacet® 9-45), glyceryl monostearate, glycerol tributyrate, polysorbate 80, polyethylene glycol, propylene glycol, oils (e.g. castor oil, hydrogenated castor oil, sesame oil, olive oil, etc.), fatty acids, and mixtures thereof. The amount of plasticizer required in the coating depends upon the nature of the plasticizer, the properties of the polymer, and the ultimate desired properties of the coating. The plasticizer may constitute from about 2% to about 50% (e.g. about 5% to about 45%) by weight of the coated particle, including, for example, about 3%, about 5%, about 7%, about 10%, about 12%, about 15%, about 17%, about 20%, about 22%, about 25%, about 27%, about 28%, about 30%, about 35%, about 40%, about 45%, and 48%, inclusive of all ranges and sub-ranges there between.

The functional polymer coating may further comprise an anti-tacking agent. Non-limiting examples of suitable anti-tacking agents include colloidal silicon dioxide, magnesium stearate, talc, glyceryl monostearate, and mixtures thereof. In one embodiment, the ratio of the coating material (functional polymer plus plasticizer) and anti-tacking agent ranges from about 9:1 to about 3:2 by weight. In another embodiment, the ratio of the coating material to anti-tacking agent ranges from about 4:1 to about 7:3 by weight.

The term “TPR coating” (also called lag-time coating) refers broadly to a coating disposed over an IR component, which delays the release of the active (e.g., tizanidine) from the TPR component. The TPR coating may include a water insoluble polymer combined with an enteric polymer, and optionally a plasticizer and anti-tacking agent. The thickness of the lag-time coating may influence the lag-time prior to release of tizanidine. In some embodiments, a barrier coating may be applied between the IR particle core and the lag-time coating layer.

In some embodiments, TPR particles are prepared by coating tizanidine-containing IR particles with a TPR coating comprising at least one functional polymer. The coating weight of a TPR coating ranges from about 5% to about 70%, including about 10% to about 40%, e.g, about 5% about 10%, about 15%, about 20%, about 25%, about 30%, about 35%, about 40%, about 45%, about 50%, about 55%, about 60%, about 65%, and about 70% w/w based on the weight of the TPR particle, inclusive of all ranges and sub-ranges therebetween. The coating weights described herein include the weight from the functional polymer (e.g., the water insoluble polymer and the enteric polymer), and, when present, the plasticizer and/or the anti-tacking agent.

In preferred embodiments, the TPR coating comprises a combination of at least one water-insoluble polymer (as described herein) and at least one enteric polymer (as described herein). The ratio of the at least one water-insoluble polymer to the at least one enteric polymer may range from about 9:1 to about 1:3, about 4:1 to about 1:2, about 4:1 to about 2:3, about 4:1 to about 1:1, about 7:3 to about 1:1, including about 9:1, about 8:1, about 7:1, about 6:1, about 5:1, about 4:1, about 3:1, about 2:1, about 1:1, about 1:2, and about 1:3 inclusive of all ranges and subranges therebetween. In preferred embodiments, the ratio of ratio of the water-insoluble polymer to the enteric polymer ranges from about 3:1 to about 1:2. In some embodiments, the ratio of the at least one water-insoluble polymer to the at least one enteric polymer is in the range of from about 45/45 to about 65/25, inclusive of all values and subranges therebetween (e.g. 45/40, 50/40, 50/35, 55/30, 60/25). In some embodiments, the TPR coating is applied to the tizanidine-containing IR particles for a weight gain in the range of from about 5% to about 70%, or from about 10% to about 60% by weight (e.g. about 5%, about 10%, about 15%, about 20%, about 25%, about 30%, about 40%, about 45%, about 50%, about 55%, about 60%, about 65%, and about 70%, inclusive of all values and subrange therebetween). The coating composition and amount of coating may be varied to provide the first TPR particles (TPR-1) and second TPR particles (TPR-2). For example, according to certain preferred embodiments, first TPR particles (TPR-1) comprising a coating amount in the range of from about 15%-35%, and the second TPR particles (TPR-2) comprises a coating amount in the range of from about 35%-55%.

A higher relative content of the water-insoluble polymer to enteric polymer in the TPR coating or a higher coating level (i.e., thickness) will result in a longer lag-time. To further modulate drug release, a barrier coating comprising a water-insoluble polymer and/or an enteric polymer, may be applied over IR particles (e.g. beads such as DIFFUCAPS® beads) prior to the TPR coating.

Thus, the release profile of the TPR component may be modulated by:

1. a barrier coating over the IR component; and/or

2. the content of the TPR coating.

The term “barrier coating” refers broadly to a coating disposed over an IR component or a TPR component to modulate drug release rate following lag-time upon oral administration. In embodiments, the barrier coating comprises a water-insoluble polymer (as described herein), a water-soluble polymer (as described herein), or an enteric polymer (as described herein), or combinations thereof. In some embodiments, the barrier coating may include a plasticizer (as described herein). In embodiments, the barrier coating is applied for a weight gain of from about 1% to about 30%, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, and about 30%, inclusive of all values and subranges therebetween.

In some embodiments, the barrier coating comprises a water-insoluble polymer (described herein) in combination with a water-soluble polymer (described herein). In such embodiments, the weight ratio of water-insoluble polymer to water-soluble polymer in the barrier coating varies from about 95:5 to about 50:50 (e.g., about 95:5, about 90:10, about 85:15, about 80:20, about 75:25, about 70:30, about 65:35: about 60:40, about 55:45, and about 50:50, inclusive of all values and subranges therebetween). In some embodiments, the barrier coating is applied to the TPR component for a weight gain of about 2% to about 20% (e.g., about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, and about 20%, inclusive of all values and subranges therebetween).

In embodiments, the barrier coating comprises an enteric polymer (as described herein) for a weight gain of from about 1% to about 25%, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, and about 25%, inclusive of all values and subranges therebetween. The enteric coating can be used alone or in combination with the water-insoluble polymer, in combination with the water-soluble polymer, or in combination with both a water-insoluble and water-soluble polymer.

Examples of barrier coating compositions include: EC 10 cPs (EC-10): HPMC E5 LV:TEC or EC-10/PEG 400 or EC-10/TEC or HPMCP (HP-55):DEP. Specific examples include: EC/HPMC E5/TEC at about 60/30/10 at about 2%-10% w/w (e.g., about 2%, about 4%, about 6%, about 8% and about 10% w/w); EC-10/PEG 400 at about 70/30 at about 2%- 10% w/w (e.g., about 2%, about 4%, about 6%, about 8%,and about 10%); or EC-10/TEC at about 90/10 at 4-10%; or HP 55/DEP at 90/10 for a weight gain of 10%, 15%, 20% by weight.

In some embodiments, the particles of the pharmaceutical compositions described herein optionally further comprise a seal coat. The term “seal coat” refers to a protective membrane disposed over a drug-containing core particle. The seal coat protects the particle from abrasion and attrition during handling. In one embodiment, the seal coat comprises a hydrophilic polymer. The seal coat may conveniently be applied at a coating weight of about 1% to about 10%, for example about 1%, about 2%, about 3%, about 4%, about 5%, about 6%, about 7%, about 8%, about 9%, and about 10%, inclusive of all ranges and sub-ranges therebetween.

Non-limiting examples of suitable hydrophilic polymers which can be used as a seal-coat or sealant coating include hydrophilic hydroxypropylcellulose (e.g., KLUCEL® LF), hydroxypropyl methylcellulose or hypromellose (e.g., OPADRY® CLEAR or PHARMACOAT™ 603), low-viscosity ethylcellulose, and mixtures thereof.

Examples of TPR coating compositions include: EC-10:HP-55:TEC or DEP over IR beads or Barrier (HP-55:DEP at about 90/10 for about 5-25% by weight) coated IR beads. Different EC-10/HP-55/DEP at a weight ratio of, for example, about 45/40/15, about 50/40/10, about 55/30/15, and about 60/25/15 may conveniently be used, and insoluble polymer is predominant. The amount of the pH dependent polymer can be decreased to increase the lag-time. Specific examples include: HP 55/EC 10/TEC at about 40:45:15; HP-55/EC-10/TEC at about 30/60/10 over IR beads (20-25 mesh) for a weight gain of about 46%; EC-10/HP-55/TEC at about 65/25/10 for a weight gain of about 45%; or EC-10/HP-55/TEC at about 55/30/15 at about 45%. The coating level may be, for example, about 10%, about 15%, about 20%, about 25%, about 30%, about 35%, about 40%, about 45%, or about 50% w/w. The lag-time will increase with increasing coating level and EC-10/HP 55/TEC weight ratio. Specific examples include: about 55:30:15 for about 15%, about 25%, about 30%, about 35%, about 40%, and about 45% weight gain.

Additional examples of TPR coating compositions include: Eudragit RSPO/Eudragit L100/EudragitS100 over IR beads. Different weight ratios of Eudragit RSPO/Eudragit L100/EudragitS100 may be used, for example, of about 50/25/25 to about 75/12.5/12.5 for example, about 10%, about 15%, about 20%, about 25%, about 30%, about 35%, about 40%, about 45%, or about 50% weight gain.

In a particular embodiment, the pharmaceutical composition which provides triphasic tizanidine release (as described herein) may comprise two populations of particles, wherein one population of TPR particles (e.g., the TPR-1 described above or the TPR-2 particles described above), has an IR component comprising the drug disposed over the TPR coating. Thus, in certain embodiments, the pharmaceutical composition described herein may comprise two particles in order to provide a triphasic release profile and blood plasma concentration profile. In some embodiments, the IR component is a seal-coated IR layer comprising the drug applied as an overcoat to the TPR coating. Appropriate amounts of the two populations of particles are combined in hard gelatin capsules to produce capsules of the disclosure having desired dose strengths.

In another particular embodiment, a pharmaceutical composition which provides triphasic tizanidine release may comprise a single population of particles which comprises the IR component, the TPR-1 component, and the TPR-2 component. Such a composition may be prepared as follows: the TPR-1 component is prepared as described above, and disposed over the TPR-2 particles as a drug layer (coating), and disposed over the TPR-1 coating is a seal-coated IR drug layer as disclosed above. Appropriate amounts of TPR particles are filled into hard gelatin capsules to produce TXR capsules with desired dose strengths.

The pharmaceutical formulation may be a dosage form such as a conventional tablet, an orally disintegrating tablet (ODT), or a capsule. The total daily dose strength may vary from 8, 12, 16, 20, 24, 32, 40, and 48 mg of tizanidine (as free base or salt). For example, appropriate amounts of IR, TPR-1 and TPR-2 particle populations may be filled into hard gelatin capsules to produce different doses of the composition, e.g. comprising 12 mg, 18 mg, 24 mg, 30 mg, 36 mg, 42 mg, and/or 48 mg of tizanidine or a pharmaceutically acceptable salt thereof. Alternatively, appropriate amounts of IR, TPR-1 and TPR-2 particle populations may be blended with rapidly dispersing granules prepared per the disclosure in U.S. Pat. No. 8,545,881, and additional ODT excipients to achieve blend homogeneity, and then compressed into Tizanidine ODTs of desired dose strengths with dose proportional tablet weights.

### Lag-Time Profile

In some embodiments, the pharmaceutical compositions described herein include at least one delayed release component (e.g. a TPR component) which is formulated to have a lag time in the range of from about 1 h to about 20 h, e.g., about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, and about 17.5, about 18, about 18.5, about 19, about 19.5, and about 20 h, inclusive of all values and subranges therebetween.

In some embodiments, the pharmaceutical compositions described herein have two or more delayed release components (e.g., the first timed pulsatile release component [e.g., TPR-1] and the second timed pulsatile release component [e.g., TPR-2]). In some embodiments, the first delayed release component (e.g., TPR-1 component) is formulated to have a lag time in the range of from about 1 to about 12 h, e.g., about 1 hour, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, and about 11.5 h, inclusive of all values and subranges therebetween. In a preferred embodiment, the first delayed release component (e.g., TPR-1 component) is formulated to have a lag time in the range of from about 3 h to about 6 h, e.g., about 3, about 3.5, about 4, about 4.5, about 5, about 5.5, and about 6 h, inclusive of all values and subranges therebetween.

In some embodiments, the second delayed release component (e.g., TPR-2 component) is formulated to have a lag time in the range of from about 6 hours to about 18 hours, e.g., about 6, about 6.5, about 7, about 7.5, about 8, about 8.5, about 9, about 9.5, about 10, about 10.5, about 11, about 11.5 h, about 12 h, about 12.5 h, about 13 h, about 13.5, about 14 h, about 14.5 h, about 15 h, about 15.5 h, about 16 h, about 16.5 h, about 17 h, about 17.5 h, and about 18 hr, inclusive of all values and subranges therebetween. In a particular embodiment, the second delayed release component (e.g., TPR-2 component) is formulated to have a lag time in the range of from about 10 to about 12 hour, e.g., about 10, about 10.5, about 11, about 11.5, and about 12 h, inclusive of all values and subranges therebetween. In a preferred embodiment, the second delayed release component (e.g., TPR-2 component) is formulated to provide a lag time in the range of from about 7 h to about 11 h, e.g., about 7, about 7.5, about 8, about 8.5, about 9, about 9.5, about 10, about 10.5, and about 11 h, inclusive of all values and subranges therebetween.

### Dissolution Profile

In some embodiments, the IR component (e.g. IR particle) completely releases tizanidine or the pharmaceutically acceptable salt thereof within about 60 minutes in vivo or in vitro in conditions simulating the acidic environment of the stomach (e.g., using the USP 2-stage dissolution test disclosed below), e.g., within about 55 minutes, about 50 minutes, about 45 minutes about 30 minutes, about 25 minutes, about 20 minutes, or about 15 minutes, inclusive of all values therebetween.

In some embodiments, the modified IR component completely releases tizanidine or the pharmaceutically acceptable salt thereof within about 3 to about 6 hours (e.g., about 4 hours) in vivo or in vitro in conditions simulating the acidic environment of the stomach plus the alkaline environment of the proximal intestinal tract (using the USP 2-stage dissolution test disclosed herein), e.g., within about 6, about 5.5, about 4, about 3.5, about 3, about 2.5, about 2, or about 1.5 hours, inclusive of all values therebetween. In some embodiments, the modified IR component provides an initial release within about 1 hour, followed by followed by complete drug release from the modified IR component within a time period of about 3-6 hours (e.g., about 3-4 hours) post-dosing (as measured using the USP 2-stage dissolution test disclosed herein).

As described above, each TPR component described herein is formulated (e.g., with a TPR coating, and optionally a barrier coating, as described herein) to provide the following release characteristics: no more than about 10% of tizanidine, or a pharmaceutically acceptable salt thereof, is released during the lag-time (as described above) following contacting the TPR component with the in vitro dissolution media or upon oral administration; and the remaining tizanidine, or the pharmaceutically acceptable salt thereof, is completely released over a defined period following the lag-time as the TPR component transitions from the acidic in vitro dissolution media (as described herein) or environment of the stomach to the less acidic (about pH 5.0 or higher) in vitro dissolution media (as described herein) or the intestinal tract. The duration of release of tizanidine, or the pharmaceutically acceptable salt thereof, from such TPR component is dictated by, inter alia, the specific composition of the barrier and/or TPR coating, as well as the thicknesses of such coatings.

In some embodiments, the pharmaceutical compositions described herein include at least one delayed release component (e.g., a TPR component), which is formulated to completely release tizanidine or the pharmaceutically acceptable salt thereof over a period of from about 3 h to about 22 h after oral administration, e.g., about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, and about 22 h, inclusive of all values and subranges therebetween. In preferred embodiments, the TPR component (e.g., TPR particles) is formulated to completely release tizanidine over a period of from about 3 h to about 10 h following oral administration, e.g., about 3, about 3.5, about 4, about 4.5, about 5, about 5.5, about 6, about 6.5, about 7, about 7.5, about 8, about 8.5, about 9, about 9.5, and about 10 h, inclusive of all values and subranges therebetween.

In some embodiments, the delayed release component (e.g., the TPR component) is formulated to completely release tizanidine or the pharmaceutically acceptable salt thereof within about 2 h to about 8 h after the lag time (as described above), e.g., within about 2, about 2.5 about 3, about 3.5, about 4, about 4.5, about 5, about 5.5, about 6, about 6.5, about 7, about 7.5, or about 8 h, inclusive of all values and subranges therebetween.

In some embodiments, the pharmaceutical compositions described herein have two or more delayed release components (e.g., the first timed pulsatile release component [TPR-1] and the second timed pulsatile release component [TPR-2]). In some embodiments, the first delayed release component (e.g., TPR-1 component) is formulated to completely release tizanidine, or the pharmaceutically acceptable salt thereof, over a period of from about 1 h to about 18 h after oral administration, e.g., about 1, about 1.5, about 2.0, about 2.5, about 3.0, about 3.5, about 4.0, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, and about 18 h, inclusive of all values and subranges therebetween. In particular embodiments, the first delayed release component (e.g., TPR-1 component) is formulated to completely release tizanidine, or the pharmaceutically acceptable salt thereof, over a period of from about 4 to about 12 hours after oral administration, e.g., about 4, about 4.5, about 5, about 5.5, about 6, about 6.5, about 7, about 7.5, about 8, about 8.5, about 9, about 9.5, about 10, about 10.5, about 11, about 11.5, and about 12 h, inclusive of all values and subranges therebetween. In further particular embodiments, the first delayed release component (e.g., TPR-1 component) is formulated to completely release tizanidine or the pharmaceutically acceptable salt thereof over a period of from about 4 to about 10 hours after oral administration, e.g., about 4, about 4.5, about 5, about 5.5, about 6, about 6.5, about 7, about 7.5, about 8, about 8.5, about 9, about 9.5, and about 10 h, inclusive of all values and subranges therebetween.

In some embodiments, the second delayed release component (e.g., TPR-2 component) completely releases tizanidine, or the pharmaceutically acceptable salt thereof, over a period of from about 6 to about 24 hours after oral administration, e.g., about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, and about 24 hours, inclusive of all values and subranges therebetween. In particular embodiments, the second delayed release component (e.g., TPR-2 component) completely releases tizanidine, or the pharmaceutically acceptable salt thereof, over a period of from about 10 hours to about 22 hours after oral administration, e.g., about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, and about 22 h, inclusive of all values and subranges therebetween. In a preferred embodiment, the second TPR component (TPR-2) completely releases tizanidine or the pharmaceutically acceptable salt thereof over a period of from about 9 hours to about 20 hours after oral administration, e.g., about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, and about 19.5 h, inclusive of all values and subranges therebetween.

In a preferred embodiment, the first TPR component is formulated to completely release tizanidine, or the pharmaceutically acceptable salt thereof, over a period of from about 2 to about 8 hours following the lag time described above, e.g., about 2, about 2.5, about 3, about 3.5, about 4, about 4.5, about 5, about 5.5, about 6, about 6.5, about 7, about 7.5, and about 8 h, inclusive of all values and subranges therebetween. In another preferred embodiment, the second TPR component is formulated to completely release tizanidine, or the pharmaceutically acceptable salt thereof, over a period of from about 8 to about 14 hours following the lag time described above, e.g., about 8, about 8.5, about 9, about 9.5, about 10, about 10.5, about 11, about 11.5, about 12, about 12.5, about 13, about 13.5, and about 14 h, inclusive of all values and subranges therebetween.

In some embodiments, the pharmaceutical composition of the disclosure (e.g. presented as a capsule) comprises an IR component together with a first timed pulsatile release component (TPR-1) having a lag time of about 3 to about 5 hours and a duration of tizanidine release of about 2 to about 6 hours, thereby providing an average blood plasma concentration profile within about 80-125% of the range of about 2.0-6.0 ng/mL (including all values and subranges therebetween) over the 8-12 hours (including all values and subranges therebetween) after administration (“bedtime component”); and a second timed pulsatile release component (TPR-2) component having a lag-time of about 8-12 hours (including all values and subranges therebetween) and a duration of tizanidine release of about 8-14 hours (including all values and subranges therebetween) following the lag-time, thereby providing a plasma tizanidine concentration-time profile within about 80-125% of the range of 1.0-5.0 ng/mL (including all values and subranges therebetween) over the latter half of 24 hours (“daytime component”) following administration. In embodiments, the dose ratio of the IR and TPR-1 component (e.g., particles) to the TPR-2 component (e.g., particles) may be in the range of about 25%:75% to about 75%:25%, or about 35%:65% to about 50%:50% (including about 25:75, about 30:70, about 35:65, about 40:60, about 45:55, about 50:50, about 60:40, about 70:30, and about 75:25, inclusive of all values and subranges therebetween). In embodiments, the bedtime component is designed to release not less than about 15% (e.g., about 15%, about 20%, about 25%, about 30%, about 35%, about 40%, about 45%, or about 50%) of the total dose of tizanidine in the pharmaceutical composition in one hour, followed by complete release in about 8-12 hours (including all values and subranges therebetween) while the daytime component has a lag-time of about 8-14 hours (including all values and subranges therebetween) followed by complete release in about 10-14 hours (including all values and subranges therebetween), when in vitro dissolution tested in USP Apparatus 1 (baskets@ at 100 rpm) or 2 (paddle@ 50 rpm) using a two-stage dissolution media (i.e., testing in 700 mL of 0.1N HCl at 37°±0.5° C. for 2 hours and thereafter at pH 6.8) and HPLC methodology. In some embodiments, the patient has a higher blood plasma tizanidine concentration during the first half of the 24 hours following oral administration than during the second half of the 24 hours. This daily blood plasma profile allows the patient to have quality sleep for about 6 to 8 hours and perform activities of daily living in early morning hours while avoiding both unanticipated spasticity attack(s) and secondary effects, such as somnolence and sedation during the daytime.

In some embodiments, the pharmaceutical composition of the disclosure (e.g. presented as a capsule) comprises an IR component together with a timed pulsatile release component (e.g., TPR particles) which is formulated to have a lag time of about 3 to about 5 hours and a duration of tizanidine release of about 2 to about 6 hours, thereby providing an average blood plasma concentration profile within about 80-125% of the range of about 2.0-6.0 ng/mL (including all values and subranges therebetween) over the 8-12 hours (including all values and subranges therebetween) after administration (“bedtime component”). In embodiments, the dose ratio of the IR component to the TPR component 25%:75% to about 50%:50% (including about 25:75, about 30:70, about 33:67, about 35:65, about 40:60, about 45:55, and about 50:50, inclusive of all values and subranges therebetween). In embodiments, the bedtime component is designed to release not less than 15% of the total dose of tizanidine in the pharmaceutical composition in one hour, followed by complete release in about 8-12 hours (including all values and subranges therebetween), when in vitro dissolution tested in USP Apparatus 1 (baskets@ at 100 rpm) or 2 (paddle@ 50 rpm) using a two-stage dissolution media (i.e., testing in 700 mL of 0.1N HCl at 37°±0.5° C. for 2 hours and thereafter at pH 6.8) and HPLC methodology. In such embodiments, this daily blood plasma profile allows the patient to have quality sleep for about 6 to 8 hours and perform activities of daily living in early morning hours while avoiding both unanticipated spasticity attack(s) and secondary effects, such as somnolence and sedation during the morning hours.

In embodiments, the pharmaceutical composition for once-daily dosing comprises a population of modified-release (IR) particles which completely release tizanidine in about 3 to 4 hours, and one or two populations of timed, pulsatile release particles. In some embodiments, the first population of TPR particles has a lag-time of about 4 hours followed by a pseudo-zero order release of tizanidine release over about 3 to 4 hours after oral administration. In other embodiments, the second population of TPR particles has a lag-time of about 8-12 hours followed by complete tizanidine release in about 10-14 hours. In such embodiments, the patient has a higher blood plasma tizanidine concentration during the first half of the 24 hours following oral administration than during the second half of the 24 hours. This daily blood plasma profile allows the patient to have quality sleep for about 6 to 8 hours and perform activities of daily living in early morning hours while avoiding both unanticipated spasticity attack(s) and secondary effects, such as somnolence and sedation during the daytime. Thus the once-daily pharmaceutical composition of the present disclosure provides sufficient control over spasticity and the onset of secondary events during night and day, thereby improving quality of life.

In some embodiments, the composition for once-daily dosing comprises: (i) a population of IR particles providing a pseudo zero order release of tizanidine or the pharmaceutically acceptable salt thereof; (ii) a first population of TPR particles having a short lag-time (e.g., about 2-6 hours, about 3-5 hours, or about 4 hours) followed by complete tizanidine release over about 3-6 hours (e.g. about 3-5 hours) for rapid onset and maintenance of spasticity control; and (iii) a second population of TPR particles having a long lag-time (e.g., about 8-12 hours, or 10-12 hours) followed by complete tizanidine release over about 8-14 hours (e.g. 9-12 hours) or about 10-12 hours) thereby providing a lower, but effective, plasma tizanidine concentration during the second half of the 24 hours following oral administration of an appropriate tizanidine XR dose in a patient with spasticity.

In further embodiments, a first population of TPR particles (TPR-1) comprise TPR coatings which allow them to exhibit the following release characteristics: no more than about 10% of tizanidine, or the pharmaceutically acceptable salt thereof, is released from the TPR-1 particles in the first about 2 to about 6 hours (e.g. about 3 to about 5 hours) following oral administration, and the remaining tizanidine, or the pharmaceutically acceptable salt, thereof is then released from the TPR-1 particles over a defined period (which may be from about 3 to about 5 hours) when the particles transition from the very acidic environment of the stomach to the less acidic (about pH 5.0 or higher) intestinal tract. The duration of tizanidine or the pharmaceutically acceptable salt thereof, release from such TPR-1 particles is dictated by the specific composition of the coating(s) and the coating thickness.

In still further embodiments, a second population of TPR particles (TPR-2) comprise particle coatings which allow them to exhibit the following release characteristics: no more than about 10% of tizanidine, or the pharmaceutically acceptable salt thereof, is released from the TPR-2 particles in the first about 8 to about 12 hours (e.g. about 8 to about 10 hours) following oral administration, and the remaining tizanidine, or the pharmaceutically acceptable salt thereof, is then completely released from the TPR-2 particles over a defined period (which may be from about 8 to about 14 hours) when the particles transition from the very acidic environment of the stomach to the less acidic (about pH 5.0 or higher) intestinal tract. The duration of tizanidine or the pharmaceutically acceptable salt thereof release from such TPR-2 particles is dictated by the specific composition of the coating(s) and the coating thickness.

In one embodiment, a pharmaceutical composition for once-daily dosing comprises a population of IR particles (e.g. mini-tablets or beads) releasing about 15% to about 25% of the total dose in about 3-4 hours, within an hour, or within about 30 minutes, a first population of TPR particles (e.g. beads or microparticles) releasing about 20% to about 35% of the dose in about 3-5 hours, and a second population of TPR particles (e.g. beads or microparticles) providing sustained release of the remaining dose, i.e., about 40 to about 65% of the total dosage in about 10-22 hours post-dosing.

In another embodiment, a pharmaceutical composition for once-daily dosing comprises a population of IR microparticles (e.g. beads) providing complete release of the tizanidine IR dose in an hour, a first population of TPR particles (e.g. beads or microparticles) releasing tizanidine in a pseudo-zero order over about 4 to about 10 hours following a lag-time of about 4 to about 6 hours, and a second population of the TPR particles (e.g. beads or microparticles) completely releasing tizanidine over about 9 to about 12 hours following a lag-time of about 9 to about 11 hours, upon oral administration to patients before bedtime.

Dissolution testing for drug release profiles described herein is performed using USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier). Drug release with time is determined by HPLC on samples pulled at selected intervals. The quantitative in vitro release tests for the particles (e.g. IR, TPR-1, and TPR-2 beads or mini-tablets) can be performed using USP 2-stage dissolution-HPLC methodology, as discussed in various embodiments exemplified in this disclosure.

In some embodiments, the IR particles are formulated to completely release tizanidine, or the pharmaceutically acceptable salt thereof, within 30 minutes following administration as measured using USP Apparatus 1 (baskets at 100 rpm) or Apparatus 2 (paddles at 50 or 75 rpm) in 700 mL of 0.1N HCl at 37° C. for 2 hours.

In some embodiments, the m-IR particles are formulated to completely release tizanidine or the pharmaceutically acceptable salt thereof in about 3 to 4 hours following administration as measured using USP 2-stage dissolution-HPLC methodology, as discussed in various embodiments above.

In embodiments, the TPR-1 particles are formulated to have a lag-time of least two hours (e.g. about 2-6 hours, or about 3-5 hours, etc.) after administration before complete release of Tizanidine or the pharmaceutically acceptable salt thereof as measured using USP Apparatus 1 (baskets at 100 rpm) or Apparatus 2 (paddles at 50 or 75 rpm), wherein the TPR-1 particles are first held for 2 hours in 700 mL of 0.1N HCl at 37° C. followed by adjusting to pH 6.8 upon addition of 200 mL of a pH modifier. In some embodiments, tizanidine or the pharmaceutically acceptable salt thereof is completely released from the TPR-1 particles over a period of from about 2-5 hours following the lag time, e.g., in about 3-4 hours.

In embodiments, the TPR-2 particles are formulated to have a lag-time of least 8 hours (e.g., about 8-12 hours) after administration before complete release of tizanidine or the pharmaceutically acceptable salt thereof as measured using USP Apparatus 1 (baskets at 100 rpm) or Apparatus 2 (paddles at 50 or 75 rpm), wherein the TPR-2 particles are first held for 2 hours in 700 mL of 0.1N HCl at 37° C. followed by adjusting to pH 6.8 upon addition of 200 mL of a pH modifier. In some embodiments, tizanidine or the pharmaceutically acceptable salt thereof is completely released from the TPR-2 particles over a period of from about 10-12 hours following the lag time.

### Pharmacokinetic Profile

As discussed above, in various embodiments, the compositions of the disclosure administered to a patient provide a therapeutically effective blood plasma concentration of tizanidine or a pharmaceutically acceptable salt thereof for about 24 hours. In some embodiments, compositions of the disclosure are formulated to provide an average plasma blood concentration profile between 80% to 125% of the range of about 1.0 to about 6.0 ng/mL of tizanidine or a pharmaceutically acceptable salt thereof over about 24 hours.

As discussed above, the once-daily pharmaceutical composition of the present disclosure are formulated to achieve a blood plasma concentration profile of tizanidine or a pharmaceutically acceptable salt thereof over the 24 hour period following administration which is effective to maintain spasticity control while controllably modulating the onset of at least one secondary effect (e.g. sedation and/or somnolence). That is, in embodiments, the pharmaceutical composition is formulated to induce the onset of somnolence and/or sedation at bedtime and reduce the onset of somnolence/sedation during the daytime, while reducing spasticity throughout a 24 hour period following administration.

In embodiments, the pharmaceutical compositions of the disclosure are formulated to achieve average blood plasma concentrations of tizanidine, or the pharmaceutically acceptable salt thereof, within about 80% to about 125% of the range of from about 1.0 to about 6.0 ng/mL for about 24 hours following oral administration of 12-36 mg of tizanidine, or a pharmaceutically acceptable salt thereof, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween. In embodiments, the pharmaceutical compositions are formulate to achieve an average blood plasma concentration of tizanidine, or the pharmaceutically acceptable salt thereof, within about 80% to 125% of the range of from about 1.0 to 4.5 ng/mL for about 24 hours following oral administration of 12-36 mg of tizanidine, or a pharmaceutically acceptable salt thereof, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, and about 6.5 ng/mL, inclusive of all values and subranges therebetween. In embodiments, the pharmaceutical compositions are formulated to achieve an average blood plasma concentration of tizanidine, or the pharmaceutically acceptable salt thereof, within about 80% to 125% of the range of from about 1.0 to 3.5 ng/mL for about 24 hours following oral administration of 12-36 mg of tizanidine, or a pharmaceutically acceptable salt thereof, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, ab out 2.0 ng/mL, ab out 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, ab out 3.0 ng/mL, ab out 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, inclusive of all values and subranges therebetween.

In embodiments, the pharmaceutical composition maintains a minimum blood plasma concentration of tizanidine or the pharmaceutically acceptable salt thereof of at least about 1.0 ng/mL for about 22 hours following administration of 12-36 mg of tizanidine.

In some embodiments, the pharmaceutical composition are formulated to provide an average blood plasma concentration within the range of 80% to 125% of about 1.5 to 6.0 ng/mL of tizanidine over a period of about 12 hours after oral administration of 12-36 mg (e.g., 24 or 28 mg) of tizanidine, e.g., about 1.0 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween.. In embodiments, the blood plasma concentrations achieved during the first about 12 hours post bedtime dosing provide spasticity control and somnolence induced sleep.

In embodiments, the blood plasma concentrations achieved during the daytime provide for spasticity control and reduced somnolence during daytime. In some embodiments, the pharmaceutical compositions are formulated to provide daytime plasma concentrations (C_{max, daytime}) within the range of 80% to 125% of about 1.0 to 4.5 ng/mL following administration of 12-36 mg of tizanidine, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, and about 6.0 ng/mL inclusive of all values and subranges therebetween.

In embodiments, the blood plasma concentrations achieved during bedtime provide spasticity control and somnolence induced sleep or sedation induced sleep. In some embodiments, the pharmaceutical compositions are formulated to achieve an average bedtime blood plasma concentrations (Cmax, bedtime) of tizanidine, or a pharmaceutically acceptable salt thereof, within about 80% to 125% of the range of from about 3.0 to 6.0 ng/mL following administration of 12-36 mg of tizanidine, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween.

In embodiments, the pharmaceutical composition comprises at least three components (e.g., at least three populations of particles) comprising: (i) an IR component; (ii) a TPR-1 component, and (iii) a TPR-2 component. In such embodiments, the components are formulated to provide different blood plasma concentrations of skeletal muscle relaxant (e.g., tizanidine or a pharmaceutically acceptable salt thereof) which control spasticity control, while also modulating sedation and/or somnolence.

Thus, in some embodiments in which the pharmaceutical compositions are formulated for bedtime administration, the IR component achieves a blood plasma concentration (C_{max, IR}) of skeletal muscle relaxant (e.g., tizanidine or the pharmaceutically acceptable salt thereof) which provides spasticity control and induces sedation and/or somnolence; the TPR-1 component achieves a blood plasma concentration (C_{max, TPR-1}) which provides spasticity control; and the TPR-2 component achieves a blood plasma concentration (C_{max, TPR-2}) which provides spasticity control and reduced sedation and/or somnolence. That is, in various embodiments, the amount of drug released from the IR and TPR-1 component provide both spasticity control and sedation and/or somnolence for up to about the first 8-12 hours after bedtime administration, and drug release from the TPR-2 component provides spasticity control but does not cause sedation and/or somnolence for about 10-24 following bedtime administration.

Accordingly, in some such embodiments, the IR component (e.g., particle) is formulated to achieve an average blood plasma concentration (C_{max, IR}) of tizanidine or the pharmaceutically acceptable salt thereof within the range of about 80% to 125% of from about 2.0 ng/mL to about 6.0 ng/mL within about 2 hours following administration of 12-36 mg of tizanidine, e.g., about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, ab out 3.0 ng/mL, ab out 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, ab out 4.0 ng/mL, ab out 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, ab out 7.0 ng/mL, ab out 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween. In particular embodiments, the C_{max, IR} of tizanidine or the pharmaceutically acceptable salt thereof within the range of about 80% to 125% of from about 3.0 ng/mL to about 5.5 ng/mL following administration of 12-36 mg of tizanidine, e.g., about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, ab out 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, ab out 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween.

In embodiments in which the IR component is a modified IR component (“m-IR”), the m-IR component (e.g., particle) achieves an average blood plasma concentration (C_{max, m-IR}) of tizanidine or the pharmaceutically acceptable salt thereof between about 80% to 125% of the range of from about 2.0 ng/mL to about 5.5 ng/mL within about 2-4 hours following administration of about 12-36 mg (e.g., about 24 or 28 mg) of tizanidine, e.g., about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, ab out 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, ab out 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, ab out 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween.

In embodiments, the TPR-1 component is formulated to provide an average maximum blood plasma concentration (C_{max, TPR-1}) of tizanidine or the pharmaceutically acceptable salt thereof within about 80% to 125% of the range of from about 1.5 ng/mL to about 5.5 ng/mL between about 5 hours to about 10 hours following administration of about 12-36 mg (e.g., about 24 or 28 mg) of tizanidine, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween. In particular embodiments, the C_{max, TPR-1} of tizanidine or the pharmaceutically acceptable salt thereof within about 80% to 125% of the range of from about 1.5 ng/mL to about 4.5 ng/mL between about 5 hours to about 10 hours following administration of about 12-36 mg (e.g., about 24 or 28 mg) of tizanidine, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, inclusive of all values and subranges therebetween.

In embodiment, the TPR-2 component is formulated to provide an average blood plasma concentration (C_{max, TPR-2}) of tizanidine or the pharmaceutically acceptable salt thereof within about 80% to 125% of the range of from about 1.5 ng/mL to about 4.5 ng/mL between about 9 hours to about 24 hours following administration of about 12-36 mg (e.g., 24 or 28 mg) of tizanidine, e.g., about 1.0 mg/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, inclusive of all values and subranges therebetween.

In embodiments in which the pharmaceutical composition is formulated for morning administration, the C_{max, IR} of skeletal muscle relaxant (e.g., tizanidine or the pharmaceutically acceptable salt thereof) provides spasticity control and reduced sedation and/or somnolence; the C_{max, TPR-1} provides spasticity control and reduced sedation and/or somnolence; and the C_{max, TPR-2} provides spasticity control and sedation and/or somnolence. Thus, in various embodiments, the amount of drug released from the IR and TPR-1 component provides spasticity control with reduced sedation and/or somnolence for the first about 12-16 hours after morning administration, and the TPR-2 component provides both spasticity control and sedation and/or somnolence for about 12-24 hours following administration. Non-limiting examples of the pharmacokinetic parameters of such compositions are provided in the following paragraphs below.

Accordingly, in some such embodiments, the IR component (e.g., particle) is formulated to provide an average blood plasma concentration (C_{max, IR}) of tizanidine or the pharmaceutically acceptable salt thereof within about 80% to 125% of the range of from about 1.5 ng/mL to about 4.5 ng/mL following administration of about 12-36 mg (e.g., about 24 or 28 mg) of tizanidine, e.g., about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, and about 7.0 ng/mL, inclusive of all values and subranges therebetween.

In embodiments in which the IR component is a modified IR component (“m-IR”), the m-IR component (e.g., particle) achieves an average blood plasma concentration (C_{max, IR}) of tizanidine or the pharmaceutically acceptable salt thereof within the range of about 80% to 125% of from about 1.5 ng/mL to about 4.5 ng/mL within about 2-4 hours following administration of 12-36 mg (e.g., about 24 or 28 mg) of tizanidine, e.g., about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, ab out 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, ab out 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, ab out 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, and about 7.0 ng/mL, inclusive of all values and subranges therebetween.

In some embodiments, the TPR-1 component is formulated to provide a C_{max, TPR-1} of tizanidine or the pharmaceutically acceptable salt thereof within about 80% to 125% of the range of from about 1.5 ng/mL to about 4.5 ng/mL between about 5 hours to about 10 hours following administration of about 1-36 mg (e.g., 24 or 28 mg) of tizanidine, e.g., about 0.5 ng/mL, about 0.6 ng/mL, about 0.7 ng/mL, about 0.8 ng/mL, about 0.9 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, and about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, ab out 5.4 ng/mL, about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, and about 7.0 ng/mL, inclusive of all values and subranges therebetween.

In some embodiment, the TPR-2 component is formulated to provide an average blood plasma concentration (C_{max, TPR-2}) of tizanidine or the pharmaceutically acceptable salt thereof within about 80% to 125% of the range of from about 2.0 ng/mL to about 6.0 ng/mL between about 9 hours to about 24 hours following administration of about 12-36 mg (e.g., 24 or 28 mg) of tizanidine, e.g., about 1.5 ng/mL, about 1.6 ng/mL, about 1.7 ng/mL, about 1.8 ng/mL, about 1.9 ng/mL, about 2.0 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, about 7.1 ng/mL, about 7.2 ng/mL, about 7.3 ng/mL, about 7.4 ng/mL, about 7.5 ng/mL, about 7.6 ng/mL, about 7.7 ng/mL, about 7.8 ng/mL, about 7.9 ng/mL, and about 8.0 ng/mL, inclusive of all values and subranges therebetween. In particular embodiments, C_{max, TPR-2} of tizanidine or the pharmaceutically acceptable salt thereof within about 80% to 125% of the range of from about 3.0 ng/mL to about 5.0 ng/mL following administration of about 12-36 mg (e.g., 24 or 28 mg) of tizanidine, e.g., about 2.3 ng/mL, about 2.4 ng/mL, about 2.5 ng/mL, about 2.6 ng/mL, about 2.7 ng/mL, about 2.8 ng/mL, about 2.9 ng/mL, about 3.0 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4.0 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5.0 ng/mL, about 5.1 ng/mL, about 5.2 ng/mL, about 5.3 ng/mL, about 5.4 ng/mL, and about 5.5 ng/mL, about 5.6 ng/mL, about 5.7 ng/mL, about 5.8 ng/mL, about 5.9 ng/mL, about 6.0 ng/mL, about 6.1 ng/mL, about 6.2 ng/mL, about 6.3 ng/mL, about 6.4 ng/mL, about 6.5 ng/mL, about 6.6 ng/mL, about 6.7 ng/mL, about 6.8 ng/mL, about 6.9 ng/mL, about 7.0 ng/mL, inclusive of all values and subranges therebetween.

### Methods of Treatment

In embodiments, the disclosure encompasses a method of treating spasticity in a patient having a neurological disease, said method comprises administering to the patient an oral pharmaceutical composition disclosed herein. In some embodiments, the method provides a blood concentration of tizanidine or pharmaceutically acceptable salt thereof that is effective for treating spasticity and also provides beneficial sedation and/or somnolence during sleep time. Generally, the treatment is administered to provide effective levels of Tizanidine, or a pharmaceutically acceptable salt thereof, to allow for about 6-8 hours of spasticity-free sleep. In embodiments, the formulations disclosed herein provide a blood concentration of at least about 1.5 ng/mL to about 6.0 ng/mL over a period of about 1-12 hours after bedtime dosing. The amount of time before bedtime that the drug may be administered will depend upon the time to reach the therapeutically effective concentration of tizanidine or the pharmaceutically acceptable salt thereof in the blood, as well as the effective duration of the formulation. As used herein, the term “effective duration” refers to the length of time that the tizanidine, or pharmaceutically acceptable salt thereof, is at an effective blood concentration level sufficient to treat spasticity, and optionally provide beneficial sedation.

In some embodiments, the disclosure also encompasses methods of improving sleep or improving sleep quality in a patient having a neurological disease, said methods comprise administering to the patient a pharmaceutical composition disclosed herein prior to bedtime to provide a blood plasma concentration of tizanidine or a pharmaceutically acceptable salt thereof of at least about 2 ng/mL or higher (e.g., about 3.0-6.0 ng/mL) over a period of several hours. As used herein, the term “improving sleep” or “improving sleep quality” means an improvement in the “Epworth sleepiness scale” as determined by a clinician.

In embodiments, the disclosure also encompasses methods for reducing daytime fatigue or sleepiness or sedation in a patient having a neurological disease comprising administering to the patient a pharmaceutical composition of the present disclosure prior to bedtime to provide a blood plasma concentration of tizanidine or a pharmaceutically acceptable salt thereof of at least about 1.0-4.0 ng/mL over a period of several hours during daytime. As used herein, the term “reducing daytime fatigue or sleepiness” means a statistically significant improvement in the “Paced Auditory Serial Addition Task” or the “Fatigue Severity Scale” as determined by a clinician.

In embodiments, the disclosure also encompasses methods for improving quality of life in a patient having a neurological disease comprising administering to the patient a pharmaceutical composition of the disclosure prior to bedtime to provide a blood plasma concentration of tizanidine or a pharmaceutically acceptable salt thereof within about 80-125% of in the range of about 3.0-6.0 ng/mL for about 6-8 hours during nighttime, and a blood plasma concentration within about 80-125% of the range of about 1.0-4.0 ng/mL for about 8-24 hours during daytime. As used herein the term “improving quality of life” or “improved quality of life” means a statistically significant increase in the score of a quality of life questionnaire as determined by a clinician.

In one embodiment, the disclosure comprises a method of treating a patient suffering from spasticity, multiple sclerosis, or amyotrophic lateral sclerosis, comprising administering a pharmaceutical composition disclosed herein before bedtime. In such embodiments, tizanidine is released in a manner which provides a blood plasma drug concentration of at least about 2 ng/mL or higher (e.g., 2-6 ng/mL) for up to 12 hours, following oral administration before bedtime, and at least about 1-4 ng/mL from about 12 hours to until next evening before bedtime dosing. The composition induces bedtime sedation by providing a slightly higher blood plasma concentration of tizanidine or the pharmaceutically acceptable salt thereof in the first 12 hours following dosing at bedtime and concomitant spasticity control to result in a patient enjoying quality sleep during the first 6 to 8 hours after dosing.

Another particular embodiment of the disclosure are oral, once-daily, pharmaceutical compositions for use in treating a neurological condition (e.g. multiple sclerosis, cerebral palsy, stroke, brain injury, or spinal cord injury) which is administered to patients at bedtime for the management of spasticity. In some embodiments, such pharmaceutical compositions comprise: (i) an IR or m-IR component providing complete release of tizanidine within about 3 to 4 hours, or within about 2 hours, or within an hour following administration; (ii) a TPR-1 component releasing tizanidine over about 4 to about 10 hours following a lag-time of about 4 to about 6 hours; and (iii) a TPR-2 component releasing Tizanidine over about 8 to about 14 hours following a lag-time of about 8 to about 10 hours.

In embodiments, the disclosure further encompasses a pharmaceutical composition for use in: (a) treating spasticity in a patient having a neurological disease; (b) improving sleep or sleep quality in a patient with a neurological disease; (c) reducing daytime fatigue or sleepiness in a patient having a neurological disease; or (d) improving daytime quality of life in a patient having a neurological disease. In some embodiments, the composition provides a blood plasma concentration of tizanidine or a pharmaceutically acceptable salt thereof in the range of about 1.0-6.0 ng/mL over a period of several hours immediately following oral administration before bedtime (e.g. about 2-5.5 ng/mL, or about 3-5.5 ng/mL over a period of about 12 hours), as well as providing a lower plasma blood concentration over a period of several hours during daytime (e.g. about 1.0-4.5 ng/mL, e.g. about 1-4.0 ng/mL, or about 1.5-4.0 ng/mL or less over a period of from about 10 to about 24 hours following oral administration before bedtime).

In some embodiments, the dissolution profiles and pharmacokinetic profiles which achieve the clinical effects described herein are provided by pharmaceutical composition for once-daily dosing comprising IR particles and TPR particles, wherein the total amount of tizanidine in the pharmaceutical composition is in the range of from about 8-48 mg (e.g., 12-36 mg). In some embodiments, the IR particles comprise from about 2 mg to about 16 mg of tizanidine, or from about 4 to about 12 mg of tizanidine, and are formulated to completely release the tizanidine with about 30 minutes to provide blood plasma levels of tizanidine which control spasticity and induce somnolence (e.g., within 80%-125% of the range of from about 5.0 ng/mL to about 6.0 ng/mL). In some embodiments, IR particles are beads comprising an inert core and an outer surface, and the tizanidine is provided in a layer on the outer surface of the inert core, or the IR particles are mini-tablets comprising tizanidine and one or more pharmaceutically acceptable excipients. In some embodiments, TPR particles comprise from about 4 mg to about 16 mg of tizanidine, and are formulated have a lag time of about 3-5 hours (e.g., about 4 hours), followed by complete release over about 5-6 hours (e.g., about 4 hours) following the lag time to provide blood plasma levels of tizanidine which control spasticity and reduce somnolence (e.g., within 80%-125% of the range of from about 3.0 ng/mL to about 5.0 ng/mL). In some embodiments, the TPR particles are beads or mini-tablets, such as the IR particles described herein, coated with a timed pulsatile release coating comprising at least one enteric polymer (as described herein) and at least one water-insoluble polymer (as described herein). In particular embodiments, the enteric polymer is a pH-sensitive methacrylic acid/methylmethacrylate copolymer, and the water-insoluble polymer is ammonium methacrylate copolymer. In some embodiments, the ratio of the at least one enteric polymer and the at least one water-insoluble polymer is within the range of from about 3:1 to about 1:2, and the timed pulsatile release coating is present at about 20% to about 45% w/w based on the weight of the TPR particle.

In some embodiments, the dissolution profiles and pharmacokinetic profiles which achieve the clinical effects described herein are provided by pharmaceutical composition for once daily dosing comprising IR particles, first TPR particles, and second TPR particles, wherein the total amount of tizanidine in the pharmaceutical composition is in the range of from about 8-48 mg (e.g., 12-36 mg). In some embodiments, the IR particles comprise from about 2 mg to about 16 mg of tizanidine, or from about 4 to about 12 mg of tizanidine, and are formulated to completely release the tizanidine with about 30 minutes to provide blood plasma levels of tizanidine which control spasticity and induce somnolence (e.g., within 80%-125% of the range of from about 5.0 ng/mL to about 6.0 ng/mL). In some embodiments, IR particles are beads comprising an inert core and an outer surface, and the tizanidine is provided in a layer on the outer surface of the inert core, or the IR particles are mini-tablets comprising tizanidine and one or more pharmaceutically acceptable excipients. In some embodiments, the first TPR particles comprise from about 4 mg to about 16 mg of tizanidine, and are formulated have a lag time of about 3-5 hours, followed by complete release over about 5-6 hours following the lag time to provide blood plasma levels of tizanidine which control spasticity and reduce somnolence (e.g., within 80%-125% of the range of from about 3.0 ng/mL to about 5.0 ng/mL). In some embodiments, the first TPR particles are beads or mini-tablets, such as the IR particles described herein, coated with a timed pulsatile release coating comprising at least one enteric polymer (as described herein) and at least one water-insoluble polymer (as described herein). In particular embodiments, the enteric polymer is a pH-sensitive methacrylic acid/methylmethacrylate copolymer, and the water-insoluble polymer is ammonio methacrylate copolymer. In some embodiments, the ratio of the at least one enteric polymer and the at least one water-insoluble polymer is within the range of from about 3:1 to about 1:2, and the timed pulsatile release coating is present at about 20% to about 45% w/w based on the weight of the first TPR particles. In some embodiments, the second TPR particles comprise from about 4 to about 16 mg of tizanidine, and are formulated have a lag time of about 8-10 hours, followed by complete release over about 9-11 hours following the lag time to provide blood plasma levels of tizanidine which control spasticity and reduce somnolence (e.g., within 80%-125% of the range of from about 2.0 ng/mL to about 5.0 ng/mL). In some embodiments, the second TPR particles are beads or mini-tablets, such as the IR particles described herein, coated with a timed pulsatile release coating comprising at least one enteric polymer (as described herein) and at least one water-insoluble polymer (as described herein). In particular embodiments, the enteric polymer is a pH-sensitive methacrylic acid/methylmethacrylate copolymer, and the water-insoluble polymer is ammonium methacrylate copolymer. In some embodiments, the ratio of the at least one water-insoluble polymer to the at least one enteric polymer is within the range of from about 3:1 to about 1:2, and the timed pulsatile release coating is present at about 35% to about 55% based on the weight of the coated particle.

All patents, patent publications, and other publications listed in this specification, are incorporated herein by reference for all purposes. While the disclosure has been described with reference to a particularly preferred embodiment, it will be appreciated that modifications can be made without departing from the spirit of the disclosure. Such modifications are intended to fall within the scope of the present disclosure.

### EXAMPLES

### Example 1

PK Plasma Profile Modeling: GastroPlus™ (Simulations Plus, Inc., Lancaster, Calif.), a physiologically based pharmacokinetic (PBPK) modeling & simulation software package, was used to simulate oral absorption, pharmacokinetics, and pharmacodynamics in humans. Pharmacokinetic parameters were optimized using the actual human plasma concentration-time data and PK model was validated against various clinical PK data (8 mg or normalized to 8 mg from other doses), such as Shah et al. Effects of Food on the Single-Dose Pharmacokinetics/Pharmacodynamics of Tizanidine Capsules and Tablets in Healthy Volunteers. Clinical Therapeutics: 28 (9), (2006); Henney et al., Relative Bioavailability of Tizanidine 4-mg Capsule and Tablet Formulations After a Standardized High-Fat Meal: A Single-Dose, Randomized, Open-Label, Crossover Study in Healthy Subjects. Clinical Therapeutics: 29 (4), (2007); Henney et al., Relative Bioavailability of Tizanidine Hydrochloride Capsule Formulation Compared with Capsule Contents Administered in

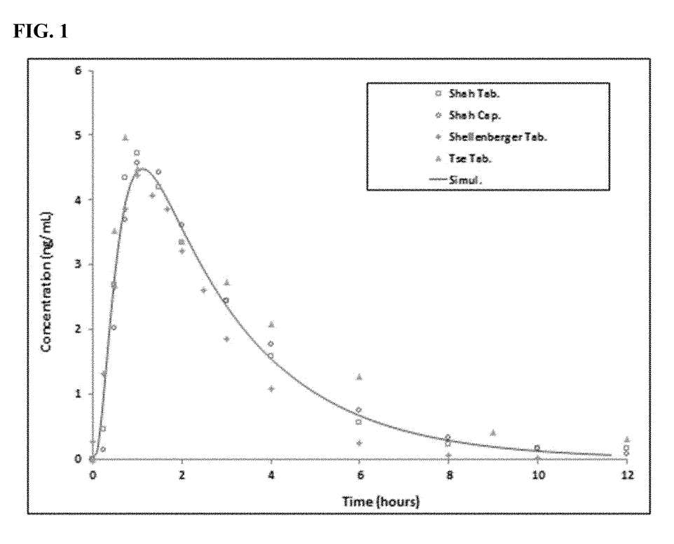

Applesauce: A Single-Dose, Open-Label, Randomized, Two-Way, Crossover Study in Fasted Healthy Adult Subjects. Clinical Therapeutics: 30 (12), (2008); Shah et al. Effects of Food on the Single-Dose Pharmacokinetics/Pharmacodynamics of Tizanidine Capsules and Tablets in Healthy Volunteers. Clinical Therapeutics: 28 (9), (2006); Shellenberger et al., A controlled pharmacokinetic evaluation of Tizanidine and baclofen at steady state. Drug Metab Dispos 1999; 27:2014; and Tse et al., Pharmacokinetics of orally administered Tizanidine in healthy volunteers. Fundam Clin Pharmacol: 1, 479-488 (1987); Zanaflex® (Tizanidine hydrochloride) [package insert]. Hawthorne, New York: Acorda Therapeutics; 2006.]. The PK model is shown in FIG. 1.

Numerous conceptual, in vitro release profiles were conceived and PK modeling and simulations were performed using the optimized PK parameters listed in Table 1 below to create triphasic plasma concentration-time profiles in subjects for the management of mild-to-moderate spasticity. Using these profiles, one can determine which profiles may be suitable, following administration at bedtime, for achieving rapid spasticity control for the first half of the 24 hours, quality sleep for about 6 to 8 hours and effective control of spasticity during the daytime, while avoiding severe side-effects, including drowsiness and sedation at work or while driving.

TABLE 1
Key parameters values for simulation

| BA | CL (L/hr/kg) | Vc (L/kg) | Thalf (hour) | Peff (×0.0001 cm/s) |
|---|---|---|---|---|
| 14% | 1.054 | 2.4122 | 1.59 | 4.7435 |

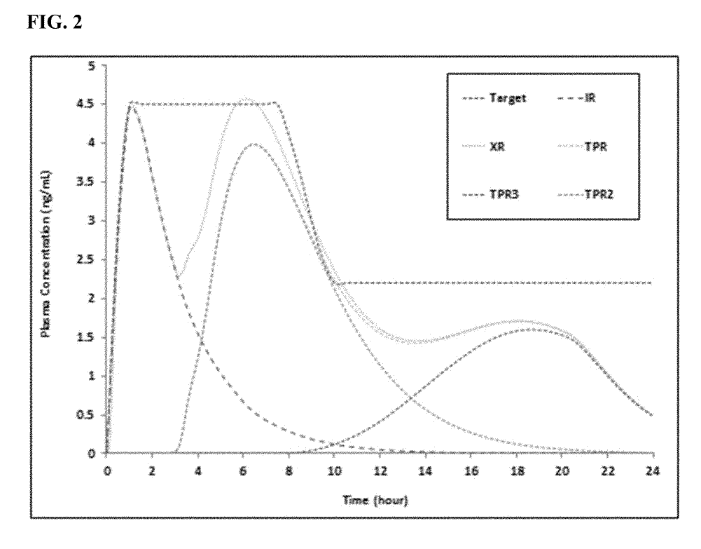

As shown in FIG. 2, the proposed target product profile (TPP) comprises three components:

First pulse: IR with 0-hour lag and 0.5-hour duration of release

Second pulse: TPR-1 with 4-hour lag and 4-hour duration of release

Third pulse: TPR-2 with 10-hour lag and 11-hour duration of release.

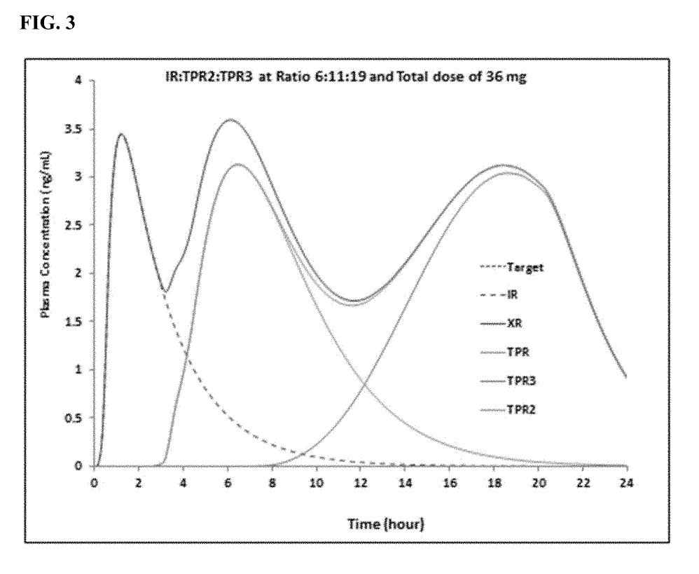

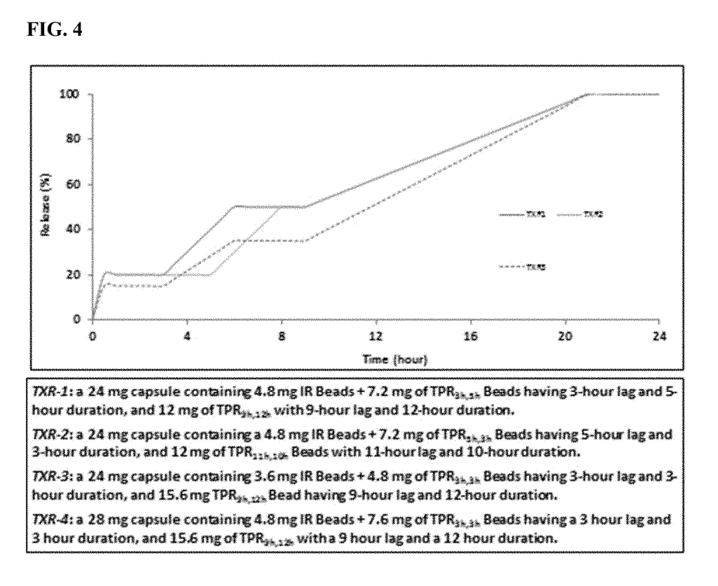

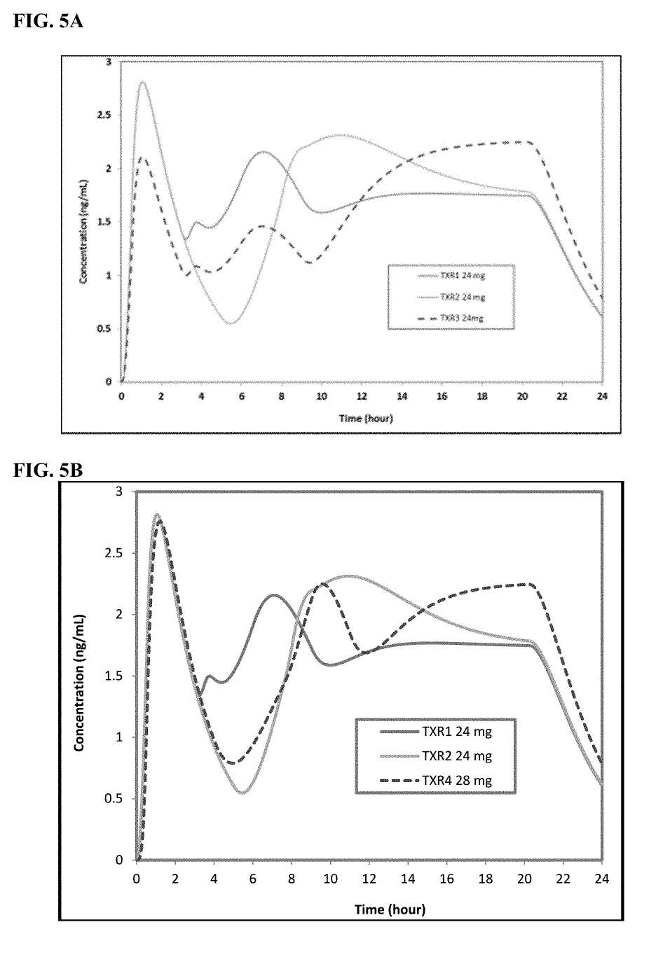

In FIG. 2, the weight ratio of IR population: TPR-1 population: TPR-2 population is 8 mg:14 mg:10 mg for a total daily dose of 32 mg. In FIG. 3, the weight ratio of IR population: TPR-1 population: TPR-2 population is 6 mg:11 mg:19 mg for a total daily dose of 32 mg. A higher Tizanidine bedtime (the first 12 hours after “before bedtime” dosing) PK profile was followed by a lower Daytime (the latter 12 hours) PK profile for a 32 mg once-daily dose. In the absence of In vitro/In vivo Correlation and extent of Tizanidine absorption from IR and TPR components along gastrointestinal tract, two scenarios were assumed to design two prototypes each comprising TPR-1 and TPR-2 with lag-times of mean±1 hour followed by pseudo-zero order Tizanidine release over mean±1 hours as shown in FIG. 4 (In vitro Tizanidine release profiles) and FIG. 5 (Simulated Tizanidine PK profiles for TXR-1, TXR-2 and TXR-3).

PK Profiles for TXR-1, TXR-2 and TXR-4 compositions:

Modified IR Particles and TXR-1 and TXR-3 Once-daily capsules

TXR-1: a 24 mg capsule containing 4.8 mg IR Beads+7.2 mg of TPR_{3h,3h} Beads having a 3 hour lag and 3 hour duration, and 12 mg of TPR_{9h,12h} Beads with a 9 hour lag and a 12 hour duration.

TXR-2: a 24 mg capsule containing a 4.8 mg IR Beads+7.2 mg of TPR_{5h,3h} Beads having a 5 hr lag and a 3 hr release duration, and 12 mg of TPR_{11h,10h} Beads with an 11 hour lag and a 10 hour release duration.

TXR-3: a 24 mg capsule containing 3.6 mg IR Beads+4.8 mg of TPR_{3h,3h} Beads having a 3 hr lag and 3 hr duration, and 15.6 mg TPR_{9h,12h} Bead having a 9 hour lag and a 12 hour release duration.

TXR-1: a 24 mg capsule containing 4.8 mg IR Beads+7.2 mg of TPR_{3h,3h} Beads having a 3 hour lag and 3 hour duration, and 12 mg of TPR_{9h,12h} Beads with a 9 hour lag and a 12 hour release duration.

TXR-2: a 24 mg capsule containing a 4.8 mg IR Beads+7.2 mg of TPR_{5h,3h} Beads having a 5 hr lag and a 3 hr duration, and 12 mg of TPR_{11h,10h} Beads with an 11 hour lag and a 10 hour release duration.

TXR-4: a 28 mg capsule containing 4.8 mg IR Beads+7.6 mg of TPR_{5h,3h} Beads having a 5 hr lag and 3 hr release duration, and 15.6 mg TPR_{11h,10h} Bead having a 11 hr lag and a 10 hr release duration.

TXR-1(IR_{0.5hr}): a 24 mg capsule containing 4.8 mg IR_{0.5hr} Beads that completely release Tizanidine within 1 hour, more preferable within 0.5 hour+7.2 mg of TPR_{3h,3h} Beads having a 3 hour lag and 3 hour release duration, and 12 mg of TPR_{9h,12h} Beads with a 9 hour lag and a 12 hour release duration.

TXR-1(IR₃₋₄ₕᵣ): a 24 mg capsule containing 4.8 mg IR₃₋₄ₕᵣ Beads that completely release Tizanidine within 3-4 hrs+7.2 mg of TPR_{3h,3h} Beads having a 3 hour lag and 3 hour duration, and 12 mg of TPR_{9h,12h} Beads with a 9 hour lag and a 12 hour release duration.

TXR-3 (IR_{0.5hr}): a 24 mg capsule containing 3.6 mg IR_{0.5hr} Beads that completely release Tizanidine within 1 hour, more preferable within 0.5 hour+4.8 mg of TPR_{3h,3h} Beads having a 3 hr lag and 3 hr release duration, and 15.6 mg TPR_{9h,12h} Bead having a 9 hour lag and a 12 hour release duration.

TXR-3 (IR₃₋₄ₕᵣ): a 24 mg capsule containing 3.6 mg IR_{0.5hr} Beads that completely release Tizanidine within 3-4 hours+4.8 mg of TPR_{3h,3h} Beads having a 3 hr lag and 3 hour release duration, and 15.6 mg TPR_{9h,12h} Bead having a 9 hour lag and a 12 hour release duration.

Based on results of the pilot Phase I randomized, double-blind, placebo-controlled, active-controlled, PK study in healthy adult volunteers, more appropriate target product profile as well as the IR to TPR-1 to TPR-2 dose ratio and multiple Once-daily TXR Capsules, such as 12 mg, 18 mg, 24 mg, 30 mg, 36 mg, 42 mg and 48 mg total dose strengths will be selected to allow dose titration and individualized patient dosing for best efficacy/tolerability profile.

### Example 2

Fluid bed Processing Tizanidine-containing Particles: GLATT™ GPCG Fluid-Bed Systems (Glatt GmbH Process Technology, Binzen, Germany) offers a series of GPCG fluid-bed coaters equipped with the twin-chamber filter system and a bottom spray Glatt HS Wurster insert, for uninterrupted processing.

### A. Tizanidine IR Beads at a Drug Load of 10% by Weight

Tizanidine hydrochloride and povidone (PVP K30) at a drug to binder weight ratio of 9:1 were dissolved in purified water at a solids content of 4% by weight and sprayed onto 25-30 mesh sugar spheres for a drug load of 10% by weight using a fluid-bed coater, GLATT GPCG 1 equipped with a 4″ bottom spray Wurster insert. The drug layering onto 25-30 mesh sugar spheres for a drug load of 10% by weight including a protective seal-coat of OPADRY CLEAR at 2% by weight was accomplished using Glatt GPCG 1 equipped with a 6″ Wurster insert.

### B. Barrier Coated IR Beads

IR beads from Example 2A. above were barrier coated with a composition comprising ethylcellulose (EC-10)/TEC at a weight ratio of 90/10 for a weight gain of up to 10%. Alternatively, EC-10/PEG 400 at a ratio of 70/30 for a weight gain of up to 10% and EC-10/HPMC E5/TEC at a weight ratio of 60/30/10 for a weight gain of up to 10% were also used to barrier coat the IR beads. In each experiment, samples were pulled after 2%, 4%, 6%, 8%, and 10% coating for dissolution testing. A barrier coating experiment with an enteric polymer (HP 55) plus TEC dissolved in 98/2 acetone/water mixture was also performed for a weight gain of 20% by weight.

### C. Preparation of TPR Beads

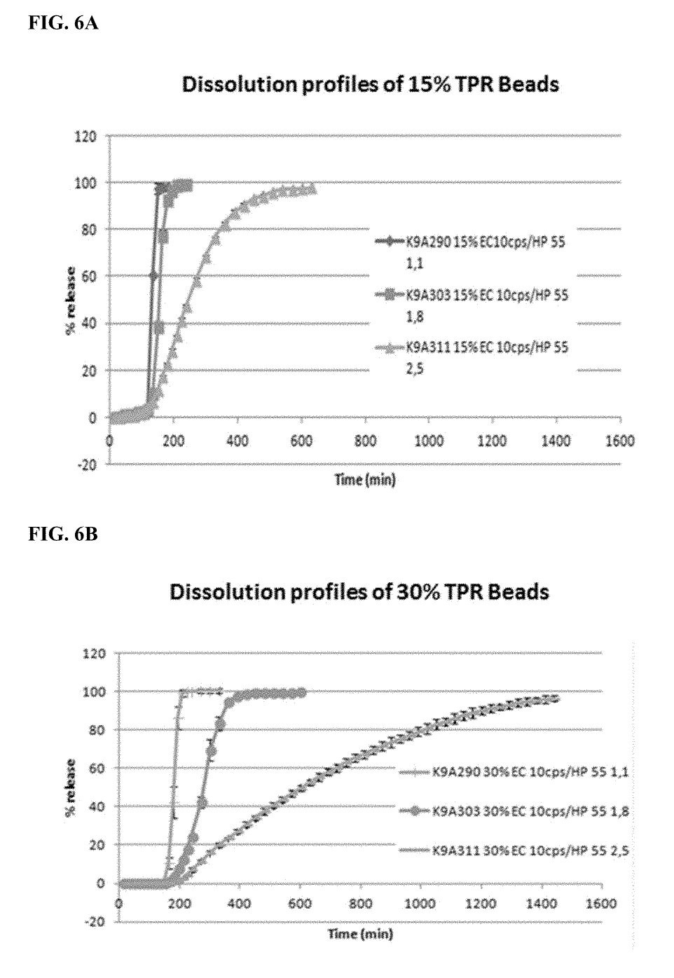

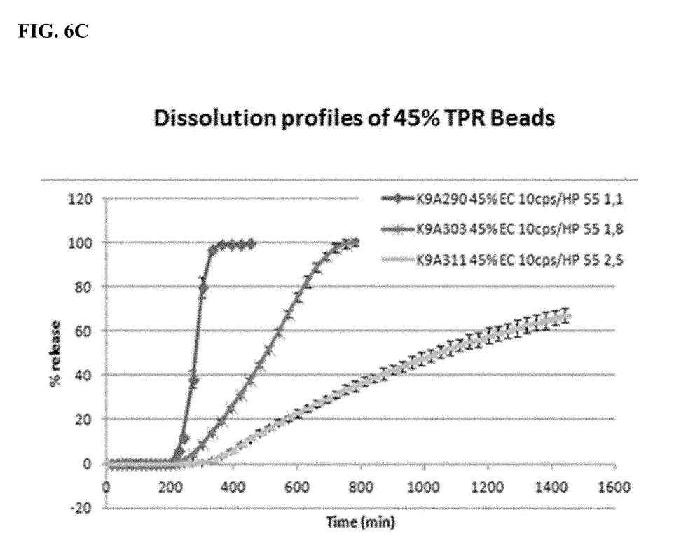

Lag-time coating formulations comprising a water-insoluble polymer [ethylcellulose (EC-10)], an enteric polymer [hypromellose phthalate (HP 55)], and a plasticizer [triethyl citrate (TEC)] at three different ratios: EC-10/HP 55/TEC of 45/40/15, 55/30/15, or 61/24/15 for a weight gain of up to 45% were prepared. IR beads from Example 2A. above were coated with these lag-time coating formulations using GPCG 1 and 6″ Wurster insert. Barrier coated IR beads from Example 2B. above were also coated with the lag-time coatings for dissolution testing. FIG. 6 shows in vitro Tizanidine release profiles for TPR beads coated with a lag-time coating with water-insoluble ethylcellulose (EC-10) in combination with enteric polymer hypromellose phthalate (HP 55) and plasticizer (triethyl citrate, TEC) at a weight ratio of 45/40/15, 55/30/15 or 61/24/15 for a weight gain of 15% (top), 30% (middle) and 45% (bottom) demonstrating the lag-times and extended release profiles achieved in each case.

### Example 3

### (i) Preparation of Tizanidine Layering Solution

Acetone (1025 g) and purified water (1025 g) were well mixed in a stainless steel container using a low shear agitator. Tizanidine hydrochloride (100.0 g) was added and mixing was continued for not less than 30 minutes until the active ingredient was completely dissolved. Povidone (11.0 g) was added to the solution and mixing was continued for not less than 30 minutes until all solids were completely dissolved.

### (ii) Preparation of Seal Coating Solution at a Solids Content of 6% by Weight

Opadry Clear YS-17006 (20.0 g) was added to 313 .3 g of water in a stainless steel container to dissolve while mixing with a low shear agitator for not less than 60 minutes.

### Step A.1: Preparation of IR Beads

Glatt GPCG-3 was set up with a 7″ Wurster insert; 20 mm partition; Nozzle height: Flush with air cap; 14 mm tubing; 100 mesh screen; Distribution plate: C; Nozzle tip size: 1.0 mm; Atomization air pressure: 1.0 bar; a dedicated filter bag with Shake interval/duration: 30 sec/5 seconds.

Method: The pre-heated GPCG 3 was charged with 16-18 mesh (1180-1000 microns) sugar spheres (869 g) and the air flow adjusted to achieve adequate fluidization. The IR coating solution prepared in Example 3(i) above was sprayed onto the sugar spheres at an initial spray rate of 6 mL/minutes, while adjusting the inlet temperature to 70° C. and airflow to 30 cfm to achieve target bed temperature of 33° C. After 10-20 minutes, the spray rate was gradually increased to 12 mL/minute while adjusting the flow parameters to maintain the target bed temperature. After the entire IR coating solution was sprayed, the seal coating solution prepared in Example 3(ii) above was sprayed on with an atomization air pressure of 1.5 bar. The seal coated beads were dried for 2 minutes, discharged and sieved through 14-mesh and 18-mesh screens to any discard agglomerates and/or fines, to provide Tizanidine IR Beads at a drug load of 10%.

### Step A.2: Preparation of IR Beads Coated with an Enteric Polymer

Hypromellose phthalate (HP-55 at 180.0 g) was added to a mixture of acetone (1620.0 g) and purified water (180.0 g) in a stainless steel container and mixed using a low shear agitator for not less than (NLT) 15 minutes (until visually dissolved). Triethyl citrate (20.0 g) was mixed for NLT 30 minutes. The preheated Glatt GPCG-3 was charged with 800 g IR beads from Step A.1 above and spray coated by spraying at an initial spray rate of 6 mL/minute; Inlet air temperature: 43° C.; Airflow: 45 cfm or so to achieve adequate fluidization and dew point: 8° C. After 10-20 minutes, the spray rate was gradually increased to 18 mL/minute by adjusting flow parameters to maintain the target bed temperature of 32-33° C. Samples were taken at a coating of 500 g, 1000 g, 1500 g and 2000 g for dissolution testing. The enteric polymer coated beads were dried, discharged, sieved through 14- and 18-mesh screens to discard over and under sized particles (“overs” and “unders”).

### Step A. 3: Preparation of TPR Beads

Hypromellose phthalate (HP-55 at 160.0 g) and EC-10 (293.3 g of ethylcellulose) were slowly added to a mixture of acetone (6376.8 g) and purified water (708.5 g) in a stainless steel container and mixed using a low shear agitator for NLT 30 minutes (until visually dissolved). Triethyl citrate (80.0 g) was mixed for NLT 30 minutes. The preheated Glatt GPCG-3 was charged with 800 g beads from Step A.2 above and spray coated by spraying at an initial spray rate of 6 mL/minute; Inlet air temperature: 41° C.; Airflow: 42 cfm or so to achieve adequate fluidization and dew point: 8° C. After 10-20 minutes, the spray rate was gradually increased to 11 mL/minute by adjusting flow parameters to maintain the target bed temperature of 32-33° C. Samples were taken at a coating of 20%, 25%, 30%, 35%, and 40% for dissolution testing. TPR beads were dried, discharged, sieved through 14- and 18-mesh screens to discard overs and unders.

### Step A.4: Additional 20% Coated TPR Beads

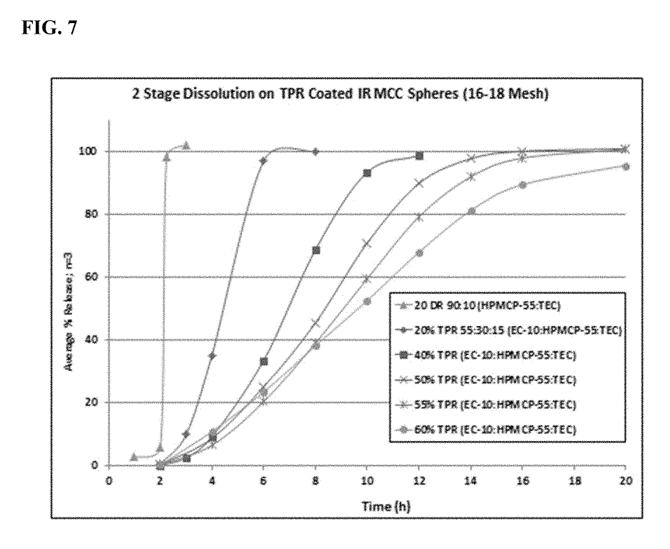

TPR beads (800 g) from Step A.3 above were sprayed with the TPR coating formulation prepared as disclosed in step A.3 at 12 mL/minute for an additional 20% by weight. Samples were pulled at 45%, 50%, 55%, and 60% by weight for dissolution testing. FIG. 6A-6C shows the in vitro Tizanidine release profiles for TPR beads coated with EC-10/HP 55/TEC at 55/30/15 wherein different coating levels resulted in varying lag-times and extended release profiles. The in vitro release profile for these TPR particles is provided in FIG. 7.

### (iii) Preparation of IR Coating Solution

Tizanidine hydrochloride (100.0 g) was added to ethanol (1027.0 g) and purified water (1027.0 g) in a stainless steel container and mixed using a low shear agitator for not less than 5 minutes until Tizanidine was completely dissolved. Povidone (15.0 g) was added to the solution which was mixed for not less than 15 minutes until all solids were completely dissolved.

### Example 4

### Step B.1: Preparation of IR Beads

25-30 mesh sugar spheres (866.36 g) were coated with the IR coating solution in (iii) above at a spray rate of 6-11 mL/min while optimally fluidizing the sugar spheres under processing conditions similar to those in Step 3.A.1 above (e.g. Inlet air temp: 45° C.; Product temp: 33° C.; Fluidization air volume: 35-40 cfm; Dew point: 7° C.). Following completion of drug layering, the beads were coated with a seal coat solution as prepared in (ii) above at a solids content of 6% by weight for a weight gain of 2% to provide Tizanidine IR Beads at a drug load of 10% by weight.

Several batches of IR beads were prepared by spraying onto 25-30 mesh (710-600 μm) sugar spheres according to the aforementioned method with a Tizanidine IR coating solution [Tizanidine (100.0 g) and povidone (13.64 g)], and seal coat solution prepared as in (ii) above for a drug load of 10% by weight, including a 2% seal-coat.

### Step B.2 Tizanidine TPR (EUDRAGIT® RSPO/L100) Beads

EUDRAGIT® RSPO (157.4 g) was added to a mixture of acetone (4197.2 g) and purified water (220.9 g) in a stainless steel container and mixed using a low shear agitator for NLT 10 minutes (until visually dissolved). EUIDRAGIT® L100 (78.7 g) was mixed for NLT 10 minutes, EUDRAGIT® 5100 (78.7 g) was mixed for NLT 10 minutes and triethyl citrate (62.7 g) was mixed for NLT 10 minutes. Talc (113.4 g) was added and mixed for NLT 30 minutes and continued mixing the suspension at low speed while spraying. The preheated Glatt GPCG-3 was charged with 700 g IR beads from step B.1 above and spray coated by spraying at an initial spray rate of 8 mL/minute; Inlet air temperature: 43° C.; Airflow: 40) cfm or so to achieve adequate fluidization and dew point: 8° C. After about 10 minutes, the spray rate was gradually increased to 18 mL/minute by adjusting flow parameters to maintain the target bed temperature of 30° C. Samples were pulled after different coating for dissolution testing. The TPR beads were dried in the unit for 5 minutes and then discharged, and sieved through 18 and 25 mesh sieves to discard overs and unders.

### Step B.3:Tizanidine TPR (EUDRAGIT® RSPO/L100/S100) Coated Beads

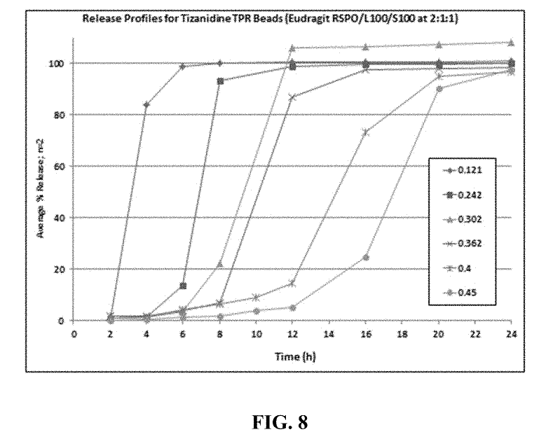

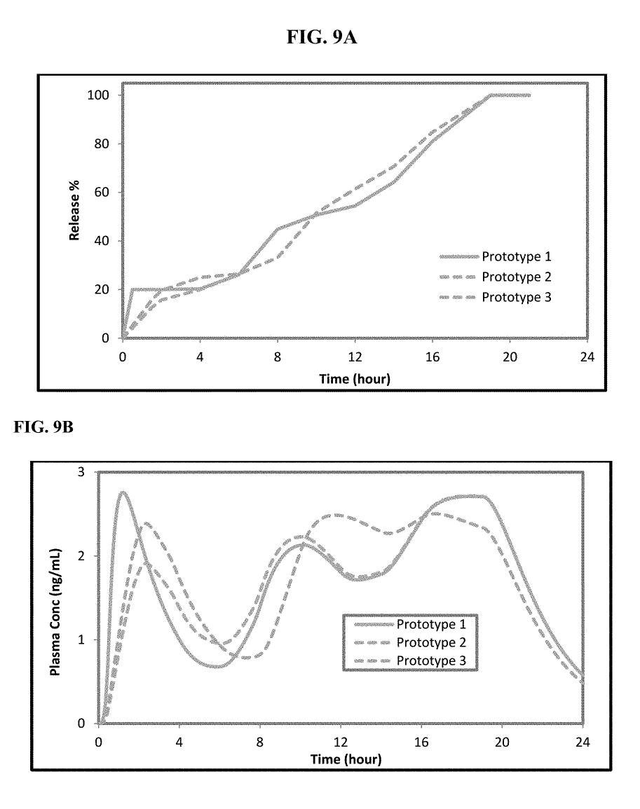

EUDRAGIT® RSPO (103.6 g) was added to a mixture of acetone (3072.0 g) and purified water (161.7 g) in a stainless steel container and mixed using a low shear agitator for NLT 10 minutes (until visually dissolved). EUDRAGIT® L100 (57.6 g) was mixed for NLT 10 minutes, EUDRAGIT® S100 (57.6 g) was mixed for NLT 10 minutes, and triethyl citrate (45.9 g) was mixed for NLT 30 minutes. Talc (83.0 g) was added and mixed for NLT 30 minutes and mixing the suspension continued at low speed while spraying. The preheated Glatt GPCG-3 was charged with 600 g IR beads from Step B.1 above and spray coated by spraying at an initial spray rate of 8-11 mL/minute; Inlet air temperature: 41° C.; Airflow: 30 cfm or so to achieve adequate fluidization and dew point: about 8° C. After about 10 minutes, the spray rate was gradually increased to 18 mL/minute by adjusting flow parameters to maintain the target bed temperature of 30-31° C. Samples were pulled after different coating for dissolution testing. The TPR beads were dried in the unit for 2 minutes and discharged. FIG. 8 shows the in vitro Tizanidine release profiles for TPR beads coated with EUDRGIT RSPO/EUDRAGIT® L100/EUDRAGIT® S100 at 2/1/1 combined with TEC and talc, wherein different coating levels resulted in varying lag-times and extended release profiles.

### Step B.4: Additional Coating of Tizanidine TPR Beads

EUDRAGIT® RSPO (42.2 g) was added to a mixture of 95/5 acetone (1125.2 g)/purified water (59.2 g) in a stainless steel container and mixed using a low shear agitator for NLT 10 minutes (until visually dissolved). EUDRAGIT® L100 (21.1 g) was mixed for NLT 10 minutes, EUDRAGIT® S100 (21.1 g) was mixed for NLT 10 minutes, and triethyl citrate (16.8 g) and talc (30.4 g) were added and mixed for NLT 30 minutes and mixing of the suspension was continued at low speed while spraying. The preheated Glatt GPCG-3 was charged with 800.0 g IR beads from Step 4.B.3 above and spray coated by spraying at an initial spray rate of 8 mL/minute; Inlet air temperature: 41° C.; Airflow: 32 cfm or so to achieve adequate fluidization and dew point: about 8° C. After about 10 minutes, the spray rate was gradually increased to 18 mL/minute by adjusting flow parameters to maintain the target bed temperature of 30-31° C. Samples were pulled after different coating for dissolution testing. The TPR beads were dried in the unit for 2 minutes, discharged, and sieved through 18 and 25-mesh sieves to discard overs (>18 mesh) and unders (<25-mesh).

### Example 5

### C.1 Tizanidine mIR (RSPO) Beads

EUDRAGIT® RSPO (210.9 g) was added to a mixture of 90/10 acetone (3044.8 g)/purified water (338.3 g) in a stainless steel container and mixed using a low shear agitator for NLT 10 minutes (until visually dissolved). Triethyl citrate (23.4 g) and talc (63.4 g) were added and mixed for NLT 30 minutes and mixing of the suspension was continued at low speed while spraying. Tizanidine IR beads (800.0 g) from Ex. 4.B.1 Step was loaded into the Glatt GPCG 3 product bowl and coated with the barrier coating formulation while maintaining adequate fluidization (e.g. spray rate of 8-18 mL/minute; Inlet air temperature: 43-45° C.; Airflow: 32 cfm; product bed temperature of 30-31° C. Following drying and discharge, the barrier coated beads were sieved through 18 and 30 mesh sieves to discard overs and unders.

### C.2 Tizanidine mIR (RSPO/RLPO) Beads

EUDRAGIT® RSPO (103.6 g) and EUDRAGIT® RLPO (103.6 g) were added to a mixture of 90/10 acetone (2762.3 g)/purified water (145.4 g) in a stainless steel container and mixed using a low shear agitator for NLT 10 minutes (until visually dissolved). Triethyl citrate (41.4 g) and talc (74.6 g) were added and mixed for NLT 30 minutes and mixing of the suspension was continued at low speed while spraying. Tizanidine IR beads (600.0 g) from Ex. 3.A.1 Step was loaded into the Glatt GPCG 3 product bowl and coated with the barrier coating formulation while maintaining adequate fluidization (e.g. spray rate of 8-18 mL/minute; Inlet air temperature: 43-45° C.; Airflow: 32 cfm; Dew point: 6-7° C.; Product bed temperature of 30-31° C. Following drying and discharge, the barrier coated beads were sieved through 18 and 30 mesh sieves to discard overs and unders.

### C. 3 Tizanidine TPR Coating (RSPO/L100/S100 at 50/20/30)

EUDRAGIT® RSPO (157.4 g) was added to a mixture of 95/5 acetone (4197.2 g)/purified water (220.9 g) in a stainless steel container and mixed using a low shear agitator for NLT 10 minutes (until visually dissolved). EUDRAGIT® L100 (63.0 g) was mixed for NLT 10 minutes, EUDRAGIT® S100 (94.4 g) was mixed for NLT 10 minutes, and triethyl citrate (62.7 g) and talc (113.4 g) were added and mixed for NLT 30 minutes and mixing of the suspension was continued at low speed while spraying. The preheated Glatt GPCG-3 was charged with IR beads (600.0 g) from Step 4.B.1 above and spray coated at a spraying rate of 8 to 18 mL/min under the following process parameters (e.g. spray rate of 8-18 mL/minute; Inlet air temperature: 41° C.; Airflow: 31-32 cfm; Dew point: 5° C.; Product bed temperature of 30-31° C.). Following drying and discharge, the TPR coated beads were sieved through 18 and 30 mesh sieves to discard overs and unders.

### C.4 Tizanidine TPR Coating (RSPO/L100/S100 at 70/15/15)

EUDRAGIT® RSPO (220.4 g) was added to a mixture of 95/5 acetone (4197.2 g)/purified water (220.9 g) in a stainless steel container and mixed using a low shear agitator for NLT 10 minutes (until visually dissolved). EUDRAGIT® L100 (47.2 g) was mixed for NLT 10 minutes, EUDRAGIT® S100 (47.2 g) was mixed for NLT 10 minutes, and triethyl citrate (62.7 g) and talc (113.4 g) were added and mixed for NLT 30 minutes and mixing of the suspension was continued at low speed while spraying. The preheated Glatt GPCG-3 was charged with IR beads (600.0 g) from Step 4.B.1 above and spray coated at a spraying rate of 8 to 18 mL/min under the following process parameters (e.g. spray rate of 8-18 mL/minute; Inlet air temperature: 42-43° C.; Airflow: 31-32 cfm; Dew point: 5° C.; Product bed temperature of 30-31° C.). Following drying and discharge, the TPR coated beads were sieved through 18 and 30 mesh sieves to discard overs and unders.

### Example 6

Appropriate amounts of IR beads or mIR beads or particles, TPR-1 particles and TPR-2 particles prepared according to the methods described herein above are combined in appropriate size hard gelatin capsules to produce once-daily capsules of the invention containing Tizanidine hydrochloride in appropriate dose strengths (e.g. 12, 18, 24, 30, 36, 42, and 48 mg).

### Example 7

A Phase I study is performed in two parts to evaluate the pharmacokinetics and pharmacodynamics and safety of the present tizanidine HCl capsules. Part A tests pharmacokinetics of three distinct timed pulse bead formulations. Part B tests pharmacokinetics, pharmacodynamics and safety of one or two complete capsule compositions compared to 8 mg Zanaflex® capsules administered every 8 hours and placebo in healthy volunteers.

In Part A, the three distinct timed pulsatile bead formulations under investigate are:

(a) Prototype A—Tizanidine Extended Release 8 mg (1 capsule). Prototype A is a capsule formulation providing only the intended 2^{nd} pulse, which consists of 8 mg tizanidine in a timed pulsatile release particle (TPR-1). Capsules are size 1, white opaque capsules containing one type of TPR bead identified as Pulse 2 generating a specific target profile of a 4-hour lag with complete release occurring approximately 5 hours after the initial 4-hour lag.

(b) Prototype B—Tizanidine Extended Release 8 mg and Immediate Release 8 mg (1 capsule). Prototype B is a capsule formulation providing the intended for the 1^{st} pulse, which contains 8 mg tizanidine (Immediate Release Component), and the intended 3^{rd} pulse, which consists of 8 mg tizanidine in a timed release particle (TPR-2). Capsules are size 1, white opaque capsules containing two types of beads: 8 mg of immediate release beads identified as Pulse 1 and 8 mg of a TPR bead identified as Pulse 3 generating a specific target profile of a 9-hour lag with complete release occurring approximately 10 hours after the initial 9-hour lag

(c) Prototype C—Tizanidine Extended Release 16 mg (1 capsule). Prototype C is a capsule formulation that provides the intended 3^{rd} pulse, which contains 16 mg of tizanidine in a timed release particle (TPR-2). Capsules are size 1, white opaque capsules containing one type of extended-release bead identified as Pulse 3 generating a specific target profile of a 9-hour lag with complete release occurring approximately 10 hours after the initial 9-hour lag.

In Part B, the compositions under investigation are:

(a) Prototype D—Tizanidine HCl ER Capsule 24 mg (Pulses 1, 2 and 3; 1 capsule)

(b) Prototype E—Tizanidine HCl ER Capsule 24 mg (Pulses 1, 2 and 3; 1 capsule; ratio of tizanidine to provide pulses 1, 2, and 3 to be determined)

(c) Placebo Capsule (1 capsule)

(d) Zanaflex Capsules. Two (2)×4 mg capsules (8 mg total), individually over-encapsulated to match the appearance of the other 3 or 4 treatments

As used in this example, “extended release” refers to the timed pulsatile release particles of the present disclosure.

Formulations used in the Phase I study are presented below in Table 2-4.

TABLE 2
Tizanidine Immediate Release Bead Final Composition

| Component | Composition % w/w |
|---|---|
| Sugar Spheres NF (25-30 mesh) | 83.06 |
| Tizanidine HCl USP | 13.00 |
| Povidone USP | 1.94 |
| Opadry Clear | 2.00 |
| Total | 100.0 |

TABLE 3
Tizanidine 25% TPR Bead (Pulse 2) Final Composition

| Component | Composition % w/w |
|---|---|
| Tizanidine Immediate Release Bead | 75.00 |
| Ammonium Methacrylate Copolymer, | 8.02 |
| Type B, NF (Eudragit RSPO) | |
| Methacrylic Acid Copolymer, Type A, | 4.01 |
| NF (Eudragit L100) | |
| Methacrylic Acid & Methyl Methacrylate | 4.01 |
| Copolymer, NF (Eudragit S100) | |
| Talc USP | 5.78 |
| Triethyl Citrate NF | 3.19 |
| Total | 100.0 |

TABLE 4
Tizanidine 45% TPR Bead (Pulse 3) Final Composition

| Component | Composition % w/w |
|---|---|
| Tizanidine Immediate Release Bead | 55.00 |
| Ammonium Methacrylate Copolymer, | 14.45 |
| Type B, NF (Eudragit RSPO) | |
| Methacrylic Acid Copolymer, Type A, | 7.20 |
| NF (Eudragit L100) | |
| Methacrylic Acid & Methyl Methacrylate | 7.20 |
| Copolymer, NF (Eudragit S100) | |
| Talc USP | 10.39 |
| Triethyl Citrate NF | 5.76 |
| Total | 100.0 |

Additional timed pulsatile release beads having a 45% TPR coating but with ratios of the polymeric components different from those reported in Table 4 (75:15:15 vs. 50:25:25 as disclosed in Table 4) were prepared to test the effect on lag time and on extended release. This TPR coating may also be applied at 30%, 35%, 40% wt based on the coated TPR bead.

Prototypes (Tizanidine ER Capsules) were prepared by encapsulating the different beads prepared according to the present disclosure. More specifically, capsules were prepared to provide different combinations of the following: Pulse 1 (IR, Tizanidine Immediate Release Bead); Pulse 2 (TPR, Tizanidine 25% TPR Coated Bead); and Pulse 3 (TPR, Tizanidine 45% (or 35%) TPR Coated Bead). These pharmaceutical compositions are presented below in Tables 5-8.

TABLE 5
Tizanidine ER Capsule 8 mg - Prototype A Final Composition

| Component | Composition % w/w |
|---|---|
| Tizanidine 25% TPR Bead | 55.3 |
| Hard Gelatin Capsule, Size 1 | 44.7 |
| Total | 100.0 |

TABLE 6
Tizanidine IR/ER Capsule 16 mg - Prototype B Final Composition

| Component | Composition % w/w |
|---|---|
| Tizanidine Drug Layered Bead | 25.7 |
| Tizanidine 45% TPR Bead | 46.6 |
| Hard Gelatin Capsule, Size 1 | 27.7 |
| Total | 100.0 |

TABLE 7
Tizanidine ER Capsule 16 mg - Prototype C Final Composition

| Component | Composition % w/w |
|---|---|
| Tizanidine 45% TPR Bead | 77.1 |
| Hard Gelatin Capsule, Size 1 | 22.9 |
| Total | 100.0 |

TABLE 8
Tizanidine ER Capsule 24 mg - Prototype D Final Composition

| Component | Composition % w/w |
|---|---|
| Tizanidine Drug Layered Bead | 19.2 |
| Tizanidine 25% TPR Bead | 25.5 |
| Tizanidine 45% TPR Bead | 34.7 |
| Hard Gelatin Capsule, Size 1 | 20.6 |
| Total | 100.0 |

TABLE 9
Tizanidine Placebo Capsule Final Composition

| Component | Composition % w/w |
|---|---|
| Sugar Spheres NF | 79.4 |
| Hard Gelatin Capsule, Size 1 | 20.6 |
| Total | 100.0 |

### Primary Objectives and Endpoints

Part A: Part A is performed to characterize the pharmacokinetics (PK) parameters for 3 distinct timed pulse bead formulations of tizanidine HCl extended release (ER) capsules. Prototype A has a single 25% timed-pulsatile release (TPR) component (2^{nd} Pulse) formulated at a dose of 8.0 mg (see Table 2). Prototype B contains the intended 1^{st} pulse with 8 mg tizanidine (Immediate Release Component) and the extended release beads for the intended 3^{rd} pulse with 8 mg tizanidine. Prototype C is the intended 3^{rd} pulse containing 16 mg tizanidine. PK assessments (Cₘₐₓ, Tₘₐₓ, AUC parameters) are determined for each of the 3 tizanidine prototype formulations (Prototypes A, B and C).

Part B: Part B is performed to characterize the PK parameters for 1 or 2 complete formulations of tizanidine HCl ER capsules (Prototype D and E; See Table 8), each consisting of an immediate release component (Pulse 1) and 2 extended release components (Pulses 2 and 3), each of which provides delayed release of tizanidine HCl at a specified time after ingestion for a specific duration after onset of release. PK assessments (Cₘₐₓ, Tₘₐₓ, AUC parameters) determined for each of the 1 or 2 formulations (Prototypes D and E) given once daily at 08:00 hand active control Zanaflex capsules 8 mg given q8h at 08:00 h, 16:00 h (daytime doses) and at 24:00 h.

### Secondary Objectives and Endpoints

The secondary objectives of the study are to evaluate the safety and tolerability of 1 or 2 tizanidine HCl formulations of the disclosure compared to placebo and to an active control, Zanaflex capsules 8 mg; and to assess simple pharmacodynamic (PD) responses to 1 or 2 tizanidine HCl ER formulations, placebo, and Zanaflex capsules 8 mg using a brief battery of cognitive function assessment tests. The brief battery of cognitive function assessment tests is performed in Part B only on study drug dosing days at 1 hour pre-dose and then 1.5 hours following each of the two daytime doses (08:00 hand 16:00 h doses).

Safety and tolerability will be assessed in terms of the frequency, severity and relationship to treatment of adverse events (AEs) including laboratory abnormalities, physical examination findings, electrocardiograms (ECG), vital signs, requirement for concomitant medications, adjunctive therapies and clinical laboratory test results.

### Exploratory Endpoints

The study's exploratory endpoints will include:

1. Comparison of immediate and extended release pulses within and between each tizanidine HCl ER formulation and Zanaflex 8 mg, as follows:

In Part A, PK parameters and plasma concentration-time profiles will be compared for:
Prototype A (Pulse 2) vs. Prototype B (Pulses 1 and 3),
Prototype A (Pulse 2) vs. Prototype C (Pulse 3), and
Prototype B (Pulses 1 and 3) vs. Prototype C (Pulse 3);

In Part B, PK parameters and plasma concentration-time profiles will be compared for:
Prototype D Pulses 1, 2 and 3 vs. Prototype E Pulses 1, 2 and 3,
Prototype D Pulses 1, 2 and 3 vs. Zanaflex 8 mg every 8 hours (Q8h)×24 hours, and
Prototype E Pulses I, 2 and 3 vs. Zanaflex 8 mg every 8 hours (Q8h)×24 hours;

2. PK/PD parametric modeling will be done as the data permits

### Study Design

Part A will be a randomized, double-blind, 3-way crossover study. On each of 3 dosing days, subjects will be administered as a single oral dose of one of three treatments, Prototype A, Prototype B or Prototype C.

Pharmacokinetic (PK) and safety measures will be collected for each of the 3 treatments. Blood samples for determination of plasma tizanidine concentration will be collected in order to determine the bioavailability of the specific dosing pulses at the following time points: pre-dose (within the 30 minutes prior to dosing) and at 15, 30, 60, 90, 120, 150, 180 and 210 minutes and at 4, 5, 6, 7, 8, 9, 10, 11, 12, 13, 14, 16, 18, 20, 22 and 24 h post-dose.

Up to 15 subjects are enrolled in Part A to ensure that 12 subjects complete the study.

Part B will be a randomized, double-blind, active- and placebo-controlled 3 or 4-way crossover study. On each of 3 or 4 dosing days, subjects will be administered one of 3 or 4 treatments, Prototype D, Prototype E, Zanaflex or placebo, each consisting of two visually identical capsules. Subjects will receive the prototype tizanidine formulations at 8:00 AM and

Zanaflex or Placebo every 8 hours (Q8h) for 24 hours—i.e., at 8:00 AM, 4:00 PM (daytime doses) and at 12:00 AM.

Note: 3^{rd} dose is given 8 hours after 2^{nd} dose, at 24:00 h (i.e., end of 1^{st} day, start of 2^{nd} day). All doses are given at the specified time±15 minutes). There is at least a 72 hour washout between dosing days.

Pharmacokinetic (PK) and safety measures are collected for each of the 3 or 4 treatments. Blood samples for determination of plasma tizanidine concentration are collected in order to determine the bioavailability of the specific dosing pulses at the following time points relative to the first dose at 08:00 h: pre-dose (within the 30 minutes prior to dosing) and at 15, 30, 60, 90, 120, 150, 180 and 210 minutes and at 4, 5, 6, 7, 8, 9, 10, 11, 12, 13, 14, 16, 18, 20, 22 and 24 hours post-dose.

The timing of PK sample collection in Part B may be modified based on the plasma tizanidine concentration results from Part A of the study.

Cognitive function assessment tests, including: 1) Karolinska Sleepiness Scale—Type B, 2) Reaction Time Test from CANTAB, and 3) Bond-Lader VAS for Alertness tests, are performed at 1 hour pre-dose (prior to 1^{st} dose) and 1.5 hours post-dose following each of the two daytime Q8h doses (08:00 and 16:00 h doses).

Karolinska Sleepiness Scale (KSS)—Type B: The Karolinska Sleepiness Scale (KSS) is a nine-point scale to assess self-reported sleepiness. The scale ranges from 1 (very alert) to 9 (very sleepy/fighting sleep/effort to keep awake). (Akerstedt T, Gilberg M. Subjective and objective sleepiness in the active individual. Int J Neurosci. 1990; 52: 29-37, which is herein incorporated by reference in its entirety for all purposes.) The task lasts for approximately 2 minutes.

CANTAB Reaction Time: The CANTAB Reaction Time test provides assessments of motor and mental response speeds, as well as measures of movement time, reaction time, response accuracy and impulsivity. The test is performed on a computerized device screen. The participant must select and hold a button at the bottom of the screen. Five-choice circles are presented above the button the participant pushes. In each case, a yellow dot will appear in one of the circles, and the participant must react as soon as possible, releasing the button at the bottom of the screen, and selecting the circle in which the dot appeared. (CANTAB Reaction Time test. http://www.cambridgecognition.com/cantab/cognitive-tests/attention/reaction-time-rti/; which is herein incorporated by reference in its entirety for all purposes). The task lasts for approximately 3 minutes.

Bond-Lader Visual Analogue Scale (VAS) of Mood and Alertness: the Bond-Lader Visual Analogue Scale (VAS) of Mood and Alertness is a 16-item inventory on which subjects are asked to rate their current state on 100 mm visual analogue scales. The scale measures the following feelings: Alert/Drowsy, Calm/Excited, Strong/Feeble, Muzzy/Clear-headed, Well-coordinated/Clumsy, Lethargic/Energetic, Contented/Discontented, Troubled/Tranquil, Mentally slow/Quick-witted, Tense/Relaxed, Attentive/Dreamy, Incompetent/Proficient, Happy/Sad, Antagonistic/Amicable, Interested/Bored, and Withdrawn/Gregarious.² The task lasts for approximately 2 minutes.

Up to 40 subjects are enrolled in Part B to ensure that 32 subjects complete the study

### Study Duration

Part A: Total duration of study participation for each subject in Part A are approximately 28 days from Screening Visit (Day-14 to Day-3) to Final Study Visit/Follow-up Visit (Day 14±1 day).

Part B: Total duration of study participation for each subject in Part B will be approximately 35 days from Screening Visit (Day-14 to Day-3) to Final Study Visit/Follow-up Visit (Day 21±1 day).

The duration of the study is approximately 2.5 months from first subject first visit (FSFV) to last subject last visit (LSLV).

### Pharmacokinetic Variables

For each of the prototype formulations in Parts A and B, the following pharmacokinetic (PK) parameters are calculated using non-compartmental analysis (NCA) methods based on actual PK sample collection times: Cₘₐₓ (ng/mL) following IR and Cₘₐₓ₂ and Cₘₐₓ₃ following second and third pulses of each ER formulation (when applicable); Tₘₐₓ (h) following IR and Cₘₐₓ₂ and Cₘₐₓ₃ following the second and third pulses of each ER formulation (when applicable); T_{1/2} (h), AUC₀₋₁, and AUC_{inf}, percentage of AUC derived by extrapolation and dose-normalized AUC. These PK parameters for the formulations are compared to PK parameters for Zanaflex capsules 8 mg (2×4 mg). Plasma concentration-time profiles are also generated and displayed graphically using nominal PK sample collection times.

### Statistics

Descriptive statistics are calculated for plasma concentration data by time and formulation, peak plasma concentration (Cₘₐₓ), Tₘₐₓ, t_{1/2}, area under the plasma concentration-time curve from dosing to the last measurable concentration (AUC₀₋ₜ,), area under the plasma concentration-time curve extrapolated to infinity (AUC_{inf}), percentage of AUC_{inf} derived from extrapolation (% AUCₑₓₜᵣₐₚ) and dose-normalized AUC.

Repeated measures analysis of variance (RM ANOVA) for repeated measures, followed by the Student-Newman-Keuls (SNK) test among individual groups are used to compare Prototype A vs B vs C in Part A and for Prototype D vs Prototype E formulations in Part B. This is done using the untransformed values as well as following rank transformation (nonparametric analysis) if the normality assumptions are not met. IR Cₘₐₓ and dose-normalized AUC are likewise compared among Prototype D, Prototype E, and Zanaflex 8 mg. These are analyzed by RM ANOVA, followed by comparison of Prototype D and Prototype D each individually versus Zanaflex 8 mg using Dunnett's test. The arithmetic mean of the individual ratios are compared with 1.0 using Student's t-test.

In addition, geometric means ratios and the 90% confidence interval (CI) are calculated. These are compared to the default BE boundaries of 0.80-1.25. Where appropriate, comparisons between male and female subjects' results are made.

In Part B, PD endpoints are summarized by treatment using descriptive statistics. Each PD endpoint for Prototype D, Prototype E, Placebo and Zanaflex 8 mg will be compared using RM ANOVA.

### Embodiments

1. A pharmaceutical composition comprising a therapeutically effective amount of tizanidine, or a pharmaceutically acceptable salt thereof, comprising:
an immediate release component (IR) comprising about 10-30 wt. % of the total amount of tizanidine, or the pharmaceutically acceptable salt thereof;
a first timed pulsatile release component (TPR-1) comprising about 25-50 wt. % of the total amount of tizanidine, or the pharmaceutically acceptable salt thereof; and
a second timed pulsatile release component (TPR-2) comprising about 40-65 wt. % of the total amount of tizanidine, or the pharmaceutically acceptable salt thereof,
wherein the pharmaceutical composition is formulated to provide therapeutically effective blood plasma levels of tizandine, or a pharmaceutically acceptable salt thereof, for up to about 24 hours following administration.

2. The pharmaceutical composition according to embodiment 1, comprising about 12 mg to about 36 mg of tizanidine, or the pharmaceutically acceptable salt thereof

3. The pharmaceutical composition according to embodiments 1 or 2, wherein the pharmaceutical composition is formulated to:
a) maintain therapeutically effective blood plasma concentration of tizanidine, or the pharmaceutically acceptable salt thereof, for about 24 hours
b) provide daytime blood plasma concentrations of tizanidine, or the pharmaceutically acceptable salt thereof, associated with spasticity control and reduced somnolence; and
c) provide an average bedtime blood plasma concentration of tizanidine, or the pharmaceutically acceptable salt thereof, associated with spasticity control and somnolence.

4. The pharmaceutical composition according to embodiment 3, wherein the therapeutically effective blood plasma concentration of tizanidine, or the pharmaceutically acceptable salt thereof, for about 24 hours is within about 80% to about 125% of the range of from about 1.0 ng/mL to about 6.0 ng/mL.

5. The pharmaceutical composition according to embodiment 3, wherein the pharmaceutical composition induces somnolence within about 1 to about 2 hours of bedtime.

6. The pharmaceutical composition according to any of embodiments 3-5, wherein the average bedtime blood plasma concentration of tizanidine, or the pharmaceutically acceptable salt thereof, is within the range of about 80% to about 125% of from about 2.5 ng/mL to about 6.0 ng/mL.

7. The pharmaceutical composition according to any of embodiments 3-6, wherein the average bedtime blood plasma concentrations of tizanidine, or the pharmaceutically acceptable salt thereof, are achieved for up to about 6 to about 8 hours following administration.

8. The pharmaceutical composition according to any of embodiments 3-7, wherein the daytime blood plasma concentration of tizanidine, or the pharmaceutically acceptable salt thereof, is within about 80% to about 125% of the range of from about 0.5 ng/mL to about 4.5 ng/mL.

9. The pharmaceutical composition according to any of embodiments 3-8, wherein the daytime blood plasma concentrations of tizanidine, or the pharmaceutically acceptable salt thereof, are achieved for about 8 to about 24 hours following administration.

10. The pharmaceutical composition according to any of embodiments 1-10, wherein each of the immediate release component, the first timed pulsatile release component, and the second timed pulsatile release component are in the form of particles.

11. The pharmaceutical composition according to any of embodiments 1-10, wherein:
the immediate release particles are formulated to provide blood plasma levels of tizanidine, or the pharmaceutically acceptable salt thereof, associated with spasticity control and somnolence;
the first timed pulsatile release particles are formulated to provide blood plasma levels of tizanidine, or the pharmaceutically acceptable salt thereof, associated with spasticity control; and
the second timed pulsatile release particles are formulated to provide blood plasma levels of tizanidine, or the pharmaceutically acceptable salt thereof, associated with spasticity control and reduced somnolence relative to the immediate release particle.

12. The pharmaceutical composition according to any of embodiments 1-11, wherein:
a. the immediate release particles are formulated to provide an average maximum blood plasma level (C_{max, IR}) of tizanidine, or the pharmaceutically acceptable salt thereof, within about 80%-125% of the range of from about 2.0 ng/mL to about 6.0 ng/mL;
b. the first timed pulsatile release particles are formulated to provide an average maximum blood plasma level (C_{max, TPR-1}) about 80%-125% of the range of from about 1.5 ng/mL to about 5.5 ng/mL of tizanidine or a pharmaceutically acceptable salt thereof; and
c. the second timed pulsatile release particle are formulated to provide an average maximum blood plasma level (C_{max, TPR-2}) within 80%-125% of the range of from about 1.5 ng/mL to about 4.0 ng/mL of tizanidine or a pharmaceutically acceptable salt thereof.

13. The pharmaceutical composition according to embodiment 12, wherein:
a. the C_{max,IR} of tizanidine, or the pharmaceutically acceptable salt thereof, is within about 80%-125% of the range of from about 3.5 ng/mL to about 6.0 ng/mL within 2 hours of administration;
b. the C_{max,TPR-1} of tizanidine or the pharmaceutically acceptable salt thereof is within about 80%-125% of the range of from about 2.5 ng/mL to about 5.5 ng/mL between about 5 hours to about 10 hours of administration; and
c. the C_{max, TPR-2} of tizanidine or the pharmaceutically acceptable salt thereof is within about 80%-125% of the range of from about 2.5 ng/mL to about 4.0 ng/mL between about 9 hours to about 24 hours of administration.

14. The pharmaceutical composition according to embodiment 12, wherein:
a. the C_{max, IR} of tizanidine or the pharmaceutically acceptable salt thereof is within about 80%-125% of the range of from about 3.5 ng/mL to about 6.0 ng/mL within 2 hours of administration;
b. the C_{max, TPR-1} of tizanidine or the pharmaceutically acceptable salt thereof is within about 80%-125% of the range of from about 2.5 ng/mL to about 5.5 ng/mL between about 6 hours to about 8 hours of administration; and
c. the C_{max, TPR-2} of tizanidine or the pharmaceutically acceptable salt thereof is within about 80%-125% of range of from about 2.5 ng/mL to about 4.0 ng/mL between about 12 hours to about 24 hours of administration.

15. The pharmaceutical composition according to embodiment 12, wherein
a. the C_{max, IR} of tizanidine or the pharmaceutically acceptable salt thereof is within about 80%-125% of the range of about 3.5 mg/mL to about 5.5 ng/mL within 2 hours of administration;
b. the C_{max, TPR-1} of tizanidine or the pharmaceutically acceptable salt thereof is within the range of about 80%-125% of the range of from about 3.0 ng/mL to about 5.0 ng/mL between about 8 hours to about 10 hours of administration; and
c. the C_{max, TPR-2} of tizanidine or the pharmaceutically acceptable salt thereof is within about 80%-125% of the range of from about 3.0 ng/mL to about 4.5 ng/mL between about 10 hours to about 24 hours of administration.

16. The pharmaceutical composition according to embodiment 12, wherein:
a. the C_{max, IR} of tizanidine or the pharmaceutically acceptable salt thereof is within about 80%-125% of the range of from about 3.5 ng/mL to about 5.5 ng/mL within about 2 hours to about 3 hours of administration, and;
b. the C_{max, TPR-1} of tizanidine or the pharmaceutically acceptable salt thereof is within about 80%-125% of the range of from about 3.0 ng/mL to 4.5 ng/mL between about 6 hours to about 8 hours following administration; and
c. the C_{max, TPR-2} of tizanidine or the pharmaceutically acceptable salt thereof within the range of about 80%-125% of the range of from about 2.0 ng/mL to about 3.5 ng/mL from about 12 hours to about 24 hours following administration.

17. The pharmaceutical according to any of embodiments 1-16, wherein the immediate release component completely releases the tizanidine, or the pharmaceutically acceptable salt thereof, within about 30 minutes as measured using USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

18. The pharmaceutical composition according to any of embodiments 1-16, wherein the first timed pulsatile release component is formulated to have a lag-time in the range of from about 2 to about 6 hours (or about 3 to about 5 hours), as measured using USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

19. The pharmaceutical composition according to embodiment 18, wherein the first timed pulsatile release component completely releases the tizanidine, or the pharmaceutically acceptable salt thereof, over a period of from about 2 to about 7 hours after the lag-time, as measured using USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

20. The pharmaceutical composition according to embodiment 19, wherein at least about 50% of the tizanidine, or the pharmaceutically acceptable salt thereof, in the first timed pulsatile release particle is released from the first timed pulsatile release particle within about 4 hour to about 6 hours following the lag-time, as measured using a USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

21. The pharmaceutical composition according to any of embodiments 1-16, wherein the second timed pulsatile release component is formulated to have a lag-time in the range of about 7 to about 11 hours following administration, as measured USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

22. The pharmaceutical composition according to embodiment 21, wherein tizanidine or the pharmaceutically acceptable salt thereof in the second timed pulsatile release particle is completely released over a period of from about 8 to about 12 hours following the lag-time, as measured using a USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

23. The pharmaceutical composition according to embodiment 22, wherein the tizanidine, or the pharmaceutically acceptable salt thereof, in the second timed pulsatile release particle is completely released over the period of from about 9 to about 11 hours following the lag-time, as measuring using a USP Apparatus 1 (baskets at 100 rpm) or Apparatus 2 (paddles at 50 or 75 rpm) and two-stage dissolution methodology (first 2 hours in 700 mL of 0.1N HCl at 37° C. followed by dissolution testing at pH 6.8 obtained by the addition of 200 mL of a pH modifier).

24. The pharmaceutical composition according any of embodiments 1-23, wherein the immediate release particle is a bead comprising an inert core and an outer surface, and tizanidine or the pharmaceutically acceptable salt thereof is present as a layer on the outer surface of the inert core.

25. The pharmaceutical composition according to embodiment 24, wherein the bead further comprises a polymeric binder and/or a solubility or absorption-enhancing polymer, which are present as an admixture with the drug on the outer surface of said inert core.

26. The pharmaceutical composition according to embodiment 25, wherein the inert core is made of sugar or microcrystalline cellulose.

27. The pharmaceutical composition according any one of embodiments 10-23, wherein the immediate release particle is a mini-tablet comprising tizanidine, or the pharmaceutically acceptable salt thereof, and one or more pharmaceutically acceptable excipients.

28. The pharmaceutical composition according to embodiment 27, wherein the one or more pharmaceutically acceptable excipients are selected from the group consisting of lactose monohydrate, silicified microcrystalline cellulose, microcrystalline cellulose, pregelatinized starch, spray-dried mannitol, povidone, hypromellose, crospovidone, sodium lauryl sulfate, magnesium stearate and sodium stearyl fumarate, and combinations thereof.

29. The pharmaceutical composition according to any one of embodiments 24 to 28, wherein the bead or mini-tablet comprises a seal coating layer.

30. The pharmaceutical composition according to embodiment 29, wherein the seal coating comprises one or more hydrophilic polymers.

31. The pharmaceutical composition according to embodiment 30, wherein one or more hydrophilic polymers is hydroxypropyl methylcellulose.

32. The pharmaceutical composition according to any one of embodiments 10-31, wherein the first timed pulsatile release particle is an immediate-release particle coated with a timed pulsatile release coating.

33. The pharmaceutical composition according to embodiment 32, wherein the timed pulsatile release coating comprises an enteric polymer and a water-insoluble polymer, and optionally an anti-tacking agent and/or a plasticizer.

34. The pharmaceutical composition according to embodiment 33, wherein the enteric polymer is selected from one or more of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof.

35. The pharmaceutical composition according to embodiment 33 or 34, wherein the enteric polymer is hydroxypropyl methylcellulose phthalate.

36. A composition according to any of embodiments 33-35, wherein the plasticizer, when present, is triethyl citrate.

37. The pharmaceutical composition according to any of embodiments 33-36, wherein the water-insoluble polymer is selected from the group consisting of cellulose acetate, cellulose acetate butyrate, polyvinyl acetate, ethyl cellulose, pH-insensitive ethyl acrylate-methyl methacrylate copolymers, and combinations thereof.

38. The pharmaceutical composition according to any of embodiments 33-37, wherein the water-insoluble polymer is ethyl cellulose or pH-insensitive ethyl acrylate-methyl methacrylate copolymers.

39. The pharmaceutical composition according to any of embodiments 33-38, wherein the anti-tacking agent, when present, is talc.

40. The pharmaceutical composition according to any one of embodiments 10 to 31, wherein the first timed pulsatile release particle is an immediate-release particle, having a delayed release undercoating beneath the timed pulsatile release coating.

41. The pharmaceutical composition according to embodiment 40, wherein the delayed release undercoating comprises an enteric polymer.

42. The pharmaceutical composition according to embodiment 41, wherein the enteric polymer is hydroxypropyl methylcellulose phthalate.

43. The pharmaceutical composition according to any one of embodiments 10 to 42, wherein the first timed pulsatile release particle is an immediate-release particle bead or immediate-release particle mini-tablet, having a barrier undercoating as an undercoat beneath the timed, pulsatile release coating and/or delayed release coating.

44. The pharmaceutical composition according to any one of the embodiments, wherein the second timed, pulsatile release particle is a timed, pulsatile release particle according to any one of embodiments 32 to 43, coated with a barrier coating as an overcoat on top the timed, pulsatile release-delaying coating.

45. The pharmaceutical composition according to embodiment 44, wherein the barrier coating comprises a water-insoluble polymer and a water-soluble polymer, and optionally a plasticizer.

46. The pharmaceutical composition according to embodiment 45, wherein the water-insoluble polymer is selected from the group consisting of cellulose acetate, cellulose acetate butyrate, polyvinyl acetate, ethyl cellulose, pH-insensitive ethyl acrylate-methyl methacrylate copolymers, and combinations thereof.

47. The pharmaceutical composition according to embodiment 45, wherein the water-soluble polymer is selected from a low molecular weight polyethylene glycol and hydroxypropyl methyl cellulose.

48. The pharmaceutical composition according to any of embodiments 1-47, having a dissolution profile whereby about ⅔ of the drug is released within the first about 12 hours and about ⅓ of the drug is released within the next about 12 hours as measured using USP Apparatus 1 (baskets at 100 rpm) or Apparatus 2 (paddles at 50 or 75 rpm) and two-stage dissolution methodology wherein the pharmaceutical composition is placed in 700 mL of 0.1N HCl at 37° C. for 2 hours followed by pH 6.8.

49. A pharmaceutical composition comprising tizanidine, or a pharmaceutically acceptable salt thereof, comprising:
a modified immediate release component comprising tizanidine, or the pharmaceutically acceptable salt thereof, in an amount from about 40 wt. % to about 80 wt. % of the total weight of tizanidine, or the pharmaceutically acceptable salt thereof, in the composition; and
a first timed pulsatile release component comprising tizanidine, or the pharmaceutically acceptable salt thereof, in an amount between from 20 wt. % to about 60 wt. % of the total weight of tizanidine, or the pharmaceutically acceptable salt thereof, in the composition;

wherein:
about 50% of the tizanidine, or the pharmaceutically acceptable salt thereof, is released from the modified immediate release component within about 30 minutes following administration, and substantially all of the tizanidine, or the pharmaceutically acceptable salt thereof, is released from the modified immediate release component within about 3 to about 6 hours, and
the timed pulsatile release component has a lag-time of about 6 to about 10 hours, and release of tizanidine, or the pharmaceutically acceptable salt thereof, occurs from about 8 to about 22 hours following administration.

50. The pharmaceutical composition according to embodiment 49, wherein:
a. the modified immediate release component provides Cₘₐₓ of tizanidine, or the pharmaceutically acceptable salt thereof, within the range of about 80% to about 125% of from about 2.0 ng/mL to about 5.5 ng/mL; and
b. timed pulsatile release component provides a Cₘₐₓ of tizanidine, or the pharmaceutically acceptable salt thereof, within the range of about 80% to about 125% of from about 1.5 ng/mL to about 4.0 ng/mL.

51. The pharmaceutical composition according to embodiment 50, wherein the modified immediate release component is an immediate release particle coated with a barrier coating as described in any one of embodiments 45 to 47.

52. The pharmaceutical composition according to embodiment 51, wherein the immediate release particles are beads, pellets, granules or mini-tablets.

53. The pharmaceutical composition according to any one of embodiments 1 to 42, wherein the pharmaceutically acceptable salt of tizanidine is a hydrochloride salt.

54. The pharmaceutical composition according to embodiments 1 to 53, wherein the pharmaceutical composition is a capsule.

55. A method of treating spasticity in a patient in need thereof, comprising administering a pharmaceutical composition according to embodiments 1-54.

56. A method of treating a patient with a neurological disease, comprising administering a pharmaceutical composition according to embodiments 1-54, wherein the treating:
i. ameliorates spasticity;
ii. improves sleep or sleep quality;
iii. reduces daytime fatigue or sleepiness; and/or
iv. improves daytime quality of life.

57. The method of treating a patient with a neurological disease according to embodiment 56, wherein the neurological disease is selected from cerebral palsy, multiple sclerosis, stroke, restless leg syndrome, spinal cord injury, and traumatic brain injury.

58. A method of treating spasticity and modulating the onset of somnolence over about a 24 hour period in a patient in need thereof, comprising administering, once daily, the pharmaceutical composition of any of embodiments 1-50.

59. The method of embodiment 58, wherein the pharmaceutical dosage from is administered to a patient at bedtime.

60. The method of embodiment 59, wherein the blood plasma concentration of tizanidine, or the pharmaceutically acceptable salt thereof, during the first about 8-12 hours following administration induces somnolence and provides spasticity control.

61. The method of embodiment 60, wherein the average blood plasma concentration of tizanidine, or the pharmaceutically acceptable salt thereof, is within the range of about 80% to about 125% of from about 2.0 ng/mL to about 6.0 ng/mL.

62. The method of embodiment 59, wherein the average plasma concentration of tizanidine, or the pharmaceutically acceptable salt thereof, during a period of about 12 to about 24 hours following administration provides spasticity control and reduces somnolence.

63. The method of embodiment 62, wherein the average blood plasma concentration of tizanidine, or the pharmaceutically acceptable salt thereof, is within the range of about 80% to about 125% of from about 1.0 ng/mL and about 4.0 ng/mL.

64. The method of any of claims 55-63, wherein tizanidine, or the pharmaceutically acceptable salt hereof, is present in an amount of about 12 mg to about 42 mg.

1. A pharmaceutical composition comprising a therapeutically effective amount of tizanidine for once-daily dosing, wherein the pharmaceutical composition comprises:
an immediate release (IR) component comprising about 10-60 wt. % of the total amount of tizanidine in the pharmaceutical composition; and
a timed pulsatile release (TPR) component comprising about 20-80 wt. % of the total amount tizanidine in the pharmaceutical composition;
wherein:
the IR component is formulated to completely release the tizanidine within 30 minutes as measured using USP Apparatus 1 (baskets at 100 rpm) or Apparatus 2 (paddles at 50 or 75 rpm) in 700 mL of 0.1N HCl, and
the TPR component is formulated to have a lag time of about 3 hours to about 8 hours and completely release the tizanidine within about 2 hr to about 8 hr after the lag time, as measured using a USP Apparatus 1 (baskets at 100 rpm) or Apparatus 2 (paddles at 50 or 75 rpm) and two-stage dissolution methodology (first 2 hr in 700 mL of 0.1N HCl at 37° C. followed by dissolution testing at pH 6.8 obtained by the addition of 200 mL of a pH modifier).

2. The pharmaceutical composition of claim 1, comprising from about 8 mg to about 42 mg of tizanidine.

3. The pharmaceutical composition of claim 1 or claim 2, comprising about 12 mg to about 36 mg of tizanidine.

4. The pharmaceutical composition of any of claims 1-3, wherein the pharmaceutical composition is formulated to provide therapeutically effective blood plasma levels of tizanidine for at least about 12 hours.

5. The pharmaceutical composition of any of claims 1-4, wherein the IR component is formulated to provide therapeutically effective blood plasma levels of tizanidine associated with controlling spasticity and inducing somnolence.

6. The pharmaceutical composition of any of claims 1-5, wherein the IR component comprises from about 2 mg to about 8 mg of tizanidine.

7. The pharmaceutical composition of any of claims 1-6, wherein the IR component is formulated to provide a first maximum blood plasma concentration (C_{max-IR}) of tizanidine within about 80%-125% of the range of from about 2.0 ng/mL to about 6.0 ng/mL following administration of about 2 mg to about 8 mg of tizanidine.

8. The pharmaceutical composition of claim 7, wherein the IR component is formulated to provide a first maximum blood plasma concentration (C_{max-IR}) of tizanidine within about 80%-125% of the range of from about 3.0 ng/mL to about 6.0 ng/mL following administration of about 8 mg of tizanidine.

9. The pharmaceutical composition of claim 8, wherein the IR component is formulated to provide a first maximum blood plasma concentration (C_{max-IR}) of tizanidine within about 80%-125% of the range of from about 5.0 ng/mL to about 6.0 ng/mL following administration of about 8 mg of tizanidine.

10. The pharmaceutical composition of any of claims 1-9, wherein the IR component is formulated to provide a time to reach Cₘₐₓ (Tₘₐₓ) of tizanidine within about 80%-125% of about 1 hr to about 2 hr.

11. The pharmaceutical composition of any of claims 1-10, wherein the IR component is in the form of IR particles.

12. The pharmaceutical composition of claim 11, wherein the IR particles are beads comprising an inert core and an outer surface, and the tizanidine is provided in a layer on the outer surface of the inert core.

13. The pharmaceutical composition of claim 12, wherein the beads further comprise a polymeric binder and a solubility-enhancing polymer or an absorption-enhancing polymer, or combination thereof, which are present in an admixture with the tizanidine in the layer on the outer surface of said inert core.

14. The pharmaceutical composition of claim 12 or claim 13, wherein the inert core is made of sugar or microcrystalline cellulose.

15. The pharmaceutical composition of claim 11, wherein the IR particles are in the form of a powder blend, beads or mini-tablets comprising tizanidine and one or more pharmaceutically acceptable excipients.

16. The pharmaceutical composition of claim 15, wherein the one or more pharmaceutically acceptable excipients are selected from the group consisting of lactose monohydrate, silicified microcrystalline cellulose, microcrystalline cellulose, pregelatinized starch, spray-dried mannitol, povidone, hypromellose, crospovidone, sodium lauryl sulfate, magnesium stearate and sodium stearyl fumarate, and combinations thereof.

17. The pharmaceutical composition of any one of claims 12-16, wherein the beads or mini-tablets comprise a seal coating layer.

18. The pharmaceutical composition of claim 17, wherein the seal coating comprises one or more hydrophilic polymers selected from the group consisting of hydrophilic hydroxypropylcellulose, hydroxypropyl methylcellulose, low-viscosity ethylcellulose, and mixtures thereof.

19. The pharmaceutical composition of claim 18, wherein the hydrophilic polymer is hydroxypropyl methylcellulose.

20. The pharmaceutical composition of any of claims 1-19, wherein the TPR component is formulated to provide therapeutically effective blood plasma levels of tizanidine associated with controlling spasticity and reduced somnolence.

21. The pharmaceutical composition of any of claims 1-20, wherein the TPR component comprises from about 4 mg to about 16 mg of tizanidine.

22. The pharmaceutical composition of any of claims 1-21, wherein the TPR component is formulated to have a lag time in the range of about 3 hr to about 7 hr.

23. The pharmaceutical composition of claim 22, wherein the TPR component is formulated to have a lag time in the range of about 3 hr to about 6 hr.

24. The pharmaceutical composition of any of claims 1-23, wherein the TPR component is formulated to release the tizanidine within about 3 hr to about 7 hr following the lag time.

25. The pharmaceutical composition of any of claims 1-24, wherein the TPR component is formulated to completely release the tizanidine within about 4 hr to about 6 hr following the lag time.

26. The pharmaceutical composition of any of claims 1-25, wherein the TPR component is formulated to provide a maximum blood plasma concentration of tizanidine (C_{max, TPR-1}) of within about 80%-125% of the range of from about 2.0 ng/mL to about 6.0 ng/mL following administration of about 4 mg to about 16 mg of tizanidine.

27. The pharmaceutical composition of any of claims 1-26, wherein the TPR component is formulated to provide a maximum blood plasma concentration of tizanidine (C_{max, TPR-1}) of within about 80%-125% of the range of from about 3.0 ng/mL to about 5.0 ng/mL following administration of about 8 mg of tizanidine.

28. The pharmaceutical composition of any of claims 1-27, wherein the TPR component is formulated to provide a time to reach C_{max, TPR-1} (T_{max, TPR-1}) of tizanidine of between about 6 hr and about 10 hr after administration.

29. The pharmaceutical composition of any of claims 1-28, wherein the TPR component is in the form of particles.

30. The pharmaceutical composition of claim 29, wherein the particles are beads or mini-tablets.

31. The pharmaceutical composition of claim 30, wherein the TPR particles are IR particles coated with a timed pulsatile release coating.

32. The pharmaceutical composition of claim 31, wherein the timed pulsatile release coating comprises at least one enteric polymer and at least one water-insoluble polymer.

33. The pharmaceutical composition of claim 32, wherein the ratio of the at least one enteric polymer and the at least one water-insoluble polymer is within the range of from about 4:1 to about 1:2.

34. The pharmaceutical composition of claim 33, wherein the ratio of the at least one enteric polymer and the at least one water-insoluble polymer is within the range of from about 3:1 to about 1:2.

35. The pharmaceutical composition of any of claims 32-34, wherein the timed pulsatile release coating is present at about 10% to about 60% w/w based on the weight of the TPR particle.

36. The pharmaceutical composition of claim 35, wherein the timed pulsatile release coating is present at about 20% to about 50% w/w based on the weight of the TPR particle, 25% to about 45% w/w based on the weight of the TPR particle, or about 15% to about 35% w/w based on the weight of the TPR particle.

37. The pharmaceutical composition of any of claims 32-36 wherein the enteric polymer is selected from the group consisting of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof.

38. The pharmaceutical composition of claim 37, wherein the enteric polymer is hydroxypropyl methylcellulose phthalate or pH-sensitive methacrylic acid/methylmethacrylate copolymers.

39. The pharmaceutical composition of any of claims 32-38, wherein the water-insoluble polymer is selected from the group consisting of cellulose acetate, cellulose acetate butyrate, polyvinyl acetate, ethyl cellulose, pH-insensitive ethyl acrylate-methyl methacrylate copolymers, and combinations thereof.

40. The pharmaceutical composition of claim 39, wherein the water-insoluble polymer is ethyl cellulose or a pH-insensitive ethyl acrylate-methyl methacrylate copolymer, such as ammonium methacrylate copolymers.

41. The pharmaceutical composition of any of claims 32-40, wherein the enteric polymer is pH-sensitive methacrylic acid/methylmethacrylate copolymers and the water-insoluble polymer is an ammonium methacrylate copolymer.

42. The pharmaceutical composition of any one of claims 31-41, wherein the TPR particles further comprise a delayed release undercoating beneath the timed pulsatile release coating.

43. The pharmaceutical composition of claim 42, wherein the delayed release undercoating comprises an enteric polymer.

44. The pharmaceutical composition of claim 43, wherein the enteric polymer is selected from the group consisting of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof.

45. The pharmaceutical composition of claim 44, wherein the enteric polymer is hydroxypropyl methylcellulose phthalate or pH-sensitive methacrylic acid/methylmethacrylate copolymers.

46. The pharmaceutical composition of any of claims 42-45, wherein the delayed release undercoating is present in an amount of from 1% to about 30% w/w based on the weight of the TPR particle.

47. The pharmaceutical composition of any of claims 42-46, wherein the TPR particles further comprise a barrier undercoating beneath the delayed release undercoating.

48. The pharmaceutical composition of claim 47, wherein the barrier undercoating comprises a water insoluble polymer, a water soluble polymer, or an enteric polymer, or combinations thereof.

49. The pharmaceutical composition of claim 47 or claim 48, wherein the barrier undercoating is present in an amount of from 1% to about 25% w/w based on the weight of the TPR particle.

50. The pharmaceutical composition of any of claims 1-49, wherein the IR component and the TPR component are formulated to provide therapeutically effective blood plasma levels of tizanidine that control spasticity for about 12-16 hours

51. The pharmaceutical composition of any of claims 1-50, wherein the TPR component is a first TPR component and the composition further comprises a second TPR component.

52. The pharmaceutical composition of claim 51, wherein the second TPR component is formulated to provide therapeutically effective blood plasma levels of tizanidine associated with controlling spasticity and reduced somnolence.

53. The pharmaceutical composition of claim 51 or 52, wherein the second TPR component comprises from about 4 to about 16 mg of tizanidine.

54. The pharmaceutical composition of any of claims 51-53, wherein the second TPR component is formulated to have a lag time in the range of from about 6 h to about 12 h.

55. The pharmaceutical composition of claim 54, wherein the second TPR component is formulated to have a lag time in the range of from about 8 h to about 10 h.

56. The pharmaceutical composition of any of claims 51-55, wherein the second TPR component is formulated to release the tizanidine within about 8 h to about 14 h following the lag time.

57. The pharmaceutical composition of claim 56, wherein the second TPR component is formulated to release the tizanidine within about 9 h to about 11 h following the lag time.

58. The pharmaceutical composition of any of claims 51-57, wherein the second TPR component is formulated to provide a maximum blood plasma concentration of tizanidine (C_{max, TPR-2}) within about 80%-125% of the range of from about 1.5 ng/mL to about 6.0 ng/mL.

59. The pharmaceutical composition of any of claims 51-58, wherein the second TPR component is formulated to provide a time to reach C_{max, TPR-2} (T_{max, TPR-2}) of between about 14 h and about 22 h after administration.

60. The pharmaceutical composition of claim 59, wherein the second TPR component is formulated to provide a T_{max, TPR-2} of between about 16 h and about 20 h after administration.

61. The pharmaceutical composition of any of claims 51-60, wherein the second TPR component is in the form of particles.

62. The pharmaceutical composition of claim 61, wherein the TPR particles are IR particles coated with a timed pulsatile release coating.

63. The pharmaceutical composition of claim 62, wherein the timed pulsatile release coating comprises at least one enteric polymer and at least one water-insoluble polymer.

64. The pharmaceutical composition of claim 63, wherein the ratio of the at least one water-insoluble polymer to the at least one enteric polymer is within the range of from about 3:1 to about 1:2.

65. The pharmaceutical composition of any of claims 63, wherein the timed pulsatile release coating is present on the TPR particle within the range of from about 35% to about 75% based on the weight of the coated particle.

66. The pharmaceutical composition of any of claims 63-65, wherein the enteric polymer is selected from the group consisting of cellulose acetate phthalate, hydroxypropyl methylcellulose phthalate, hydroxypropyl methylcellulose acetate succinate, pH-sensitive methacrylic acid/methylmethacrylate copolymers, cellulose acetate phthalate, cellulose acetate phthalate aqueous dispersion, hydroxypropyl methylcellulose acetate succinate aqueous dispersion, shellac, and combinations thereof.

67. The pharmaceutical composition of claim 66, wherein the enteric polymer is hydroxypropyl methylcellulose phthalate or pH-sensitive methacrylic acid/methylmethacrylate copolymers.

68. The pharmaceutical composition of any of claims 63-67, wherein the water-insoluble polymer is selected from the group consisting of cellulose acetate, cellulose acetate butyrate, polyvinyl acetate, ethyl cellulose, pH-insensitive ethyl acrylate-methyl methacrylate copolymers, and combinations thereof.

69. The pharmaceutical composition of claim 68, wherein the water-insoluble polymer is ethyl cellulose or a pH-insensitive ethyl acrylate-methyl methacrylate copolymer, such as an ammonium methacrylate copolymer.

70. The pharmaceutical composition of any of claims 63-69, wherein the enteric polymer is pH-sensitive methacrylic acid/methylmethacrylate copolymers and the water-insoluble polymer is an ammonium methacrylate copolymer.

71. The pharmaceutical composition of any of claims 61-70, wherein the second TPR particles comprise TPR particles as claimed in claims 29-50, coated with a barrier coating applied over the timed pulsatile release coating.

72. The pharmaceutical composition of claim 71, wherein the barrier coating comprises at least one water-insoluble polymer and at least one water-soluble polymer.

73. The pharmaceutical composition of claim 72, wherein the water-insoluble polymer is selected from the group consisting of cellulose acetate, cellulose acetate butyrate, polyvinyl acetate, ethyl cellulose, pH-insensitive ethyl acrylate-methyl methacrylate copolymers, and mixtures thereof.

74. The pharmaceutical composition of claim 73 or claim 74, wherein the water-soluble polymer is selected from a low molecular weight polyethylene glycol and hydroxypropyl methyl cellulose.

75. The pharmaceutical composition of any of claims 1-74, wherein the tizanidine is in the form of a hydrochloride salt.

76. The pharmaceutical composition of any of claims 1-75, wherein the pharmaceutical composition is a capsule.

77. A method of treating spasticity in a patient in need thereof, comprising administering a pharmaceutical composition according to any of claims 1-76.

78. The method of claim 77, wherein the treating achieves at least one of the following:
i. ameliorates spasticity;
ii. improves sleep or sleep quality;
iii. reduces daytime fatigue or sleepiness; or
iv. improves daytime quality of life.

79. The method of claim 77 or 78, wherein the patient has a neurological disease.

80. The method of claim 79, wherein the neurological disease is selected from cerebral palsy, multiple sclerosis, stroke, restless leg syndrome, spinal cord injury, and traumatic brain injury.

81. A method of treating spasticity and modulating the onset of somnolence over about a 24 hour period in a patient in need thereof, comprising administering, once daily, the pharmaceutical composition of any of claims 1-76.

82. The method of claim 81, wherein the pharmaceutical composition is administered to a patient at bedtime.

83. The method of claim 82, wherein the average blood plasma concentration of tizanidine during the first about 8-10 hours following administration induces somnolence and provides spasticity control.

84. The method of claim 83, wherein the average blood plasma concentration of tizanidine is within about 80% to about 125% of the range of from about 2.0 ng/mL to about 5.5 ng/mL during the first about 8-10 hours following administration.

85. The method of claim 84, wherein the average blood plasma concentration of tizanidine during a period of about 8 to about 24 hours following administration provides spasticity control and reduced somnolence.

86. The method of claim 85, wherein the average blood plasma concentration of tizanidine is within about 80% to about 125% of the range of from about 1.0 ng/mL and about 4.0 ng/mL for about 12 to about 22 hours following administration.

87. A pharmaceutical composition for once daily dosing according to any of claims 1-76 for use in treating spasticity in a patient in need thereof.

88. Tizanidine, or a pharmaceutically acceptable salt thereof, in a medicament for use in method to treat spasticity in a patient in need thereof, wherein the tizanidine is administered in a pharmaceutical composition to according to any of claims 1-76.

89. The pharmaceutical composition of claim 87, or the tizanidine of claim 88, wherein the pharmaceutical composition is administered before bedtime.
